(19) United States
(12) Patent Application Publication
(10) Pub. No.: US 2022/0288118 A1
(43) Pub. Date: Sep. 15, 2022

(54) DEPLETION REGIMES FOR ENGINEERED T-CELL OR NK-CELL THERAPY

(71) Applicant: Forty Seven, Inc., Foster City, CA (US)

(72) Inventors: Craig Gibbs, Palo Alto, CA (US); Kristopher Marjon, Palo Alto, CA (US); Jens-Peter Volkmer, Menlo Park, CA (US)

(21) Appl. No.: 17/627,319

(22) PCT Filed: Jul. 30, 2020
(86) PCT No.: PCT/US2020/044269

§ 371 Date: Jan. 14, 2022

Related U.S. Application Data
(60) Provisional application No. 62/881,268, filed on Jul. 31, 2019.

(51) Int. Cl. A61K 35/17; C07K 14/735; C07K 16/28; A61K 45/06; A61K 39/395; A61K 38/17

(52) U.S. Cl. CPC A61K 35/17; C07K 14/70535; C07K 16/2803; A61K 45/06; A61K 39/39558; A61K 38/1774; A61K 2039/5158

(57) ABSTRACT

The invention provides method of depleting endogenous T-cells or NK-cells to facilitate propagation or survival of engineered T-cells introduced into a subject for a therapeutic purpose. The depletion regime involves a co-administration of an immunotherapeutic agent against T-cells and an immunotherapeutic agent that inhibits CD47 interaction with NK-cells. The immunotherapeutic agent against T-cells or NK-cells binds to an antigen on T-cells or NK-cells effecting depletion of the T-cells or NK-cells, which depletion is promoted by the immunotherapeutic agent inhibiting CD47-SIRPα interaction. The genetically engineered T-cells or NK-cells can have a variety of genetic modifications such as a chimeric antigen receptor that targets the T-cells to a target cell.

### CROSS-REFERENCE TO RELATED APPLICATION

The present application claims the benefit of 62/881,268, filed Jul. 31, 2019, incorporated by reference in its entirety for all purposes.

### SEQUENCE LISTING

The application includes sequences disclosed in txt file 550879SEQ of 32 kbytes created Jul. 6, 2020, which is incorporated by reference.

### BACKGROUND

Engineered T-cell therapy was developed to treat cancer with T-cells from a patient or other source, and it has been established over many years through ex vivo manipulation, expansion and infusion of T-cells. Its effectiveness is based on antigen specificity of T-cells. This specificity can be enhanced by the genetic modification and redirection of T-cells to target antigens that are overexpressed in cancers. T-cells can be engineered to express modified T-cell receptors (TCRs) (so-called TCR therapies) or Chimeric Antigen Receptors (CARs) that enhance antigen specificity. These approaches overcome central and peripheral tolerance, generating T-cells more efficient at targeting cancers without requiring de novo T-cell activation in the patient. Most patients receive lymphodepleting chemotherapy before infusion of engineered T-cells to increase proliferation of the engineered T-cells. Current methods of lymphodepletion rely on radiation and/or chemotherapy, which can impart toxic effects limiting the potential clinical utility of infused T-cells.

### SUMMARY OF THE CLAIMED INVENTION

The invention provides a method of performing T-cell or NK cell therapy in a subject in need thereof, comprising: administering to the subject a combination therapy comprising an immunotherapeutic agent antagonizing CD47 interaction with SIRPα and an immunotherapeutic agent binding to a T-cell or NK cell antigen, thereby depleting endogenous T-cells or NK-cells of the subject, wherein the subject is also administered genetically engineered T-cells or NK-cells. Optionally, the subject is administered the genetically engineered T-cells. Optionally, the T-cell are genetically engineered to have a chimeric antigen receptor. Optionally, the chimeric antigen receptor, comprises an scFv or Fab, a transmembrane domain and an intracellular signaling domain. Optionally, the chimeric antigen receptor comprises a CD16 extracellular domain, a transmembrane domain and an intracellular signaling domain, wherein the CD16 domain is complexed with an Fc domain of an antibody. Optionally, the genetically engineered T-cells are genetically engineered to express alpha and beta domains of a T-cell receptor. Optionally, the genetically engineered NK-cells are administered. Optionally, the immunotherapeutic agent antagonizing CD47 interaction with SIRPα is an antibody specifically binding to CD47, such as magrolimab. Optionally, the antibody specifically binding to CD47 is administered as a priming dose followed by a higher therapeutic dose. Optionally, the immunotherapeutic agent antagonizing CD47 interaction with SIRPα is an antibody specifically binding to SIRPα. Optionally, the antibody comprises a heavy chain variable region having a sequence comprising SEQ ID NO:19 and a light chain variable region having a sequence comprising SEQ ID NO:20. Optionally, the antibody specifically binding SIRPα is any of FSI-189, ES-004, BI765063, ADU1805, and CC-95251. Optionally, the immunotherapeutic agent antagonizing CD47 interaction with SIRPα is administered at a dose of 10-30 mg/kg. Optionally, a single dose of the antibody specifically binding to SIRPα is administered. Optionally, two or more doses of the antibody specifically binding to SIRPα are administered. Optionally, the immunotherapeutic agent specifically binding to a T-cell antigen specifically binds to CD2, CD3, CD4, CD8, CD52, CD45 or ATG. Optionally, the T-cells administered to the subject are autologous T-cells. Optionally, the T-cells administered to the subject are allogenic T-cells. Optionally, the T-cells administered to the subject have a T-cell receptor linked to an antibody against a cancer-associated antigen. Optionally, the subject has a cancer expressing the cancer-associated antigen and the T-cells or NK-cells are engineered to bind to the antigen. Optionally, the combination therapy is performed before the subject is administered the T-cells or NK-cells. Optionally, the T-cells or NK-cells administered to the subject are engineered for reduced binding to the immunotherapeutic agent specifically binding to the T-cell or NK cell antigen and/or the immunotherapeutic agent antagonizing CD47 interaction with SIRPα. Optionally, the combination therapy does not include an antibody specifically binding to c-kit. Optionally, the combination therapy does not include a genotoxic or myeloablative agent. Optionally, the combination therapy does not include dimethyl busulfan. Optionally, the subject has a cancer. Optionally, the cancer is a leukemia, lymphoma, myeloma or myelodysplastic syndrome.

### Definitions

A subject or patient includes both humans being treated by the disclosed methods and other animals, particularly mammals, including pets and laboratory animals, e.g. mice, rats, rabbits, and non-human primates. Thus the methods are applicable to both human therapy and veterinary applications.

An immunotherapeutic agent refers to an antibody or Fc-fusion protein specifically binding to a designated target. Such an immunotherapeutic agent can equivalently be described as being against the designated target. For example, antibodies against CD47 and a SIRPα-Fc fusion are immunotherapeutic agents against CD47.

Immunotherapeutic agents are typically provided in isolated form. This means that such an agent is typically at least 50% w/w pure of interfering proteins and other contaminants arising from its production or purification but does not exclude the possibility that the agent is combined with an excess of pharmaceutical acceptable carrier(s) or other vehicle intended to facilitate its use. Sometimes agents are at least 60, 70, 80, 90, 95 or 99% w/w pure of interfering proteins and contaminants from production or purification. Often an agent is the predominant macromolecular species remaining after its purification.

Specific binding of an immunotherapeutic agent to its target antigens means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces.

A basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. However, reference to a variable region does not mean that a signal sequence is necessarily present; and in fact signal sequences are cleaved once antibodies or other immunotherapeutic agents of the invention have been expressed and secreted. A pair of heavy and light chain variable regions defines a binding region of an antibody. The carboxy-terminal portion of the light and heavy chains respectively defines light and heavy chain constant regions. The heavy chain constant region is primarily responsible for effector function. In IgG antibodies, the heavy chain constant region is divided into CH1, hinge, CH2, and CH3 regions. In IgA, the heavy constant region is divided into CH1, CH2 and CH3. The CH1 region binds to the light chain constant region by disulfide and noncovalent bonding. The hinge region provides flexibility between the binding and effector regions of an antibody and also provides sites for intermolecular disulfide bonding between the two heavy chain constant regions in a tetramer subunit. The CH2 and CH3 regions are the primary site of effector functions and FcRn binding.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” segment of about 12 or more amino acids, with the heavy chain also including a “D” segment of about 10 or more amino acids. (See generally, Fundamental Immunology (Paul, W., ed., 2nd ed. Raven Press, N.Y., 1989), Ch. 7) (incorporated by reference in its entirety for all purposes).

The mature variable regions of each light/heavy chain pair form the antibody binding site. Thus, an intact antibody has two binding sites, i.e., is divalent. In natural antibodies, the binding sites are the same. However, in bispecific the binding sites are different (see, e.g., Songsivilai and Lachmann, Clin. Exp. Immunol., 79:315-321 (1990); Kostelny et al., J. Immunol., 148:1547-53 (1992)). The variable regions all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989). Kabat also provides a widely used numbering convention (Kabat numbering) in which corresponding residues between different heavy chain variable regions or between different light chain variable regions are assigned the same number. Although Kabat numbering can be used for antibody constant regions, the EU index is more commonly used, as is the case in this application.

The term “epitope” refers to a site on an antigen to which an arm of a bispecific antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. Some antibodies bind to an end-specific epitope, meaning an antibody binds preferentially to a polypeptide with a free end relative to the same polypeptide fused to another polypeptide resulting in loss of the free end. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996). When the application refers to a particular antibody, the application should be understood as also disclosing other antibodies binding to the same epitope.

Antibodies that recognize the same or overlapping epitopes can be identified in a simple immunoassay showing the ability of one antibody to compete with the binding of another antibody to a target antigen. The epitope of an antibody can also be defined X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20×. or 100×) inhibits binding of the reference antibody by at least 50% but preferably 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. When the application refers to a particular antibody, it should also be understood as also disclosing other antibodies competing with that antibody for binding to its specified target.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Nonconservative substitutions constitute exchanging a member of one of these classes for a member of another.

Percentage sequence identities are determined with antibody sequences maximally aligned by the Kabat numbering convention for a variable region or EU numbering for a constant region. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

The term “antibody-dependent cellular cytotoxicity”, or ADCC, is a mechanism for inducing cell death that depends upon the interaction of antibody-coated target T-cells (i.e., cells with bound antibody) with immune cells possessing lytic activity (also referred to as effector cells). Such effector cells include natural killer cells, monocytes/macrophages and neutrophils. ADCC is triggered by interactions between the Fc region of an antibody bound to a cell and Fcγ receptors, particularly FcγRI and FcγRIII, on immune effector cells such as neutrophils, macrophages and natural killer cells. The target cell is eliminated by phagocytosis or lysis, depending on the type of mediating effector cell. Death of the antibody-coated target cell occurs as a result of effector cell activity.

The term “antibody-dependent cellular phagocytosis,” or ADCP, refers to the process by which antibody-coated cells are internalized, either in whole or in part, by phagocytic immune cells (e.g., macrophages, neutrophils and dendritic cells) that bind to an immunoglobulin Fc region.

The term “complement-dependent cytotoxicity” or CDC refers to a mechanism for inducing cell death in which an Fc effector domain(s) of a target-bound antibody activates a series of enzymatic reactions culminating in the formation of holes in the target cell membrane. Typically, antigen-antibody complexes such as those on antibody-coated target-cells bind and activate complement component C1q which in turn activates the complement cascade leading to target cell death. Activation of complement may also result in deposition of complement components on the target cell surface that facilitate ADCC by binding complement receptors (e.g., CR3) on leukocytes.

Genotoxic regimens comprise, at least in part, the administration of agents with direct or indirect effects on the DNA: the induction of mutations, mistimed event activation, and direct DNA damage leading to mutations. Examples of genotoxic agents include radiation and certain chemotherapeutic drugs, such as alkylating agents, intercalating agents and inhibitors of enzymes involved in DNA replication. Myeloablative conditioning regimens are combination of agents expected to produce profound pancytopenia and myeloablation within 1-3 weeks from administration; pancytopenia is long lasting, usually irreversible and in most instances fatal, unless hematopoiesis is restored by hemopoietic stem cell infusion. Examples include total body irradiation and/or administration of alkylating agents; fludarabine, dimethyl busulfan, etoposide (VP16). There is significant overlap in genotoxic and myeloablative agents. The methods of the invention do not require genotoxic or myeloablative agents.

Unless otherwise apparent from the context reference to a range should be understood as also disclosing all subranges defined by integers within the range.

Any dosage or dosage range provided herein in mg/kg can be converted to an absolute dosage in mg using an exemplary human body weight of 70 kg, optionally with rounding of the dosage, or upper and lower bounds of the dosage to the nearest integer, or nearest 10, 50, 100, 500 or 1000 integer encompassing the calculated absolute dose. Thus, for example, a dosage range of 0.15-2 mg/kg can be converted to 10.5 to 140 mg, or with exemplary rounding, 10 to 150 mg. Likewise a dosage range of 10-30 mg/kg can be converted to 700-2100 mg, or with exemplary rounding 500-2500 mg.

### BRIEF DESCRIPTION OF THE FIGURES

FIG. 1: CAR in which CD16 (Fc gamma receptor) is used as a universal adaptor for an antibody.

FIG. 2: Structures of first, second, and third generation CARs and TRUCKs.

### DETAILED DESCRIPTION

I. General

The invention provides methods of depleting endogenous T-cells or NK-cells to facilitate propagation or survival of engineered T-cells or NK cells introduced into a subject for a therapeutic purpose. Although practice of the invention is not dependent on an understanding of mechanism, it is believed the depletion of endogenous T-cells or NK-cells upregulates cytokine production and decreases competition for cytokines between endogenous and engineered T-cells or NK-cells, increasing propagation and persistence of the engineered T-cells or NK-cells. The depletion regime involves a co-administration of an immunotherapeutic agent against T-cells or NK-cells and an immunotherapeutic agent that inhibits CD47 interaction with SIRPα. The immunotherapeutic agent against T-cells or NK-cells binds to an antigen on T-cells or NK-cells effecting depletion of the T-cells or NK-cells, which depletion is promoted by the immunotherapeutic agent inhibiting CD47-SIRPα interaction. Such a regime does not require genotoxic or myoablative agents, nor does it require an immunotherapeutic agent against c-kit. Use of immunotherapeutic agent against c-kit in combination with an immunotherapeutic agent inhibiting CD47-SIRPα interaction is described in PCT/US2020/034049, filed May 21, 2020 incorporated by reference in its entirety for all purposes. The genetically engineered T-cells or NK-cells can have a variety of genetic modifications such as a chimeric antigen receptor that targets the T-cells to a target cell.

II. Immunotherapeutic Agents Depleting T-Cells or NK-Cells

Immunotherapeutic agents can bind to and deplete T-cells, NK-cells or both. Such agents typically bind to an antigen having an extracellular domain displayed on the surface of T-cells or NK-cells, or both. Such agents can act by a mechanism such as ADCC, ADPC, CDC, apoptosis, antagonism of target interactions with a ligand or co-receptor, or toxicity of a conjugated drug. Immunotherapeutic agents that target T-cells include, for example, antibodies specific for CD2, CD3, CD4, CD8, CD52 (campath), CD45, and anti-thymocyte globulin (ATG). Immunotherapeutic agents against CD2 and CD52 also target NK-cells. Immunotherapeutic agents that selectively target NK-cells include, for example, antibodies against CD122 and CD56.

Multiple anti-human CD3 mAb are in clinical development, including teplizumab, and MGA031, is a humanized IgG1 antibody that was developed by grafting the complementarity determining region of OKT3 into a human IgG1 backbone. Otelixizumab (ChAglyCD3, TRX4, GSK2136525) is derived from the rat antibody YTH12.5, and is a humanized IgG1 with a single mutation in the yl Fc portion to avoid glycosylation and thus inhibit FcR binding. Visilizumab (Nuvion, HuM291) is a humanized IgG2 antibody rendered non mitogenic by two point mutations in its Fc region. Foralumab (28F11-AE; NI-0401) is an entirely human anti-CD3 mAb.

An exemplary anti-CD52 antibody is the clinically approved antibody Campath (alemtuzumab), which is a recombinant DNA-derived humanized monoclonal antibody directed against the 21-28 kDa cell surface glycoprotein, CD52. Campath-1H is an IgG1 kappa antibody with human variable framework and constant regions, and complementarity-determining regions from a murine (rat) monoclonal antibody (Campath-1G). Campath can be administered, for example, at the currently accepted clinical dose, e.g. escalating to the maximum single dose of 30 mg over a period of from about 3 to about 7 days.

Antibody-based therapy can use monoclonal (e.g., muromonab-CD3 and anti-CD25 antibodies (e.g., basiliximab, daclizumab), or polyclonal, for example, an ATG preparation, aKT3, BTI-322© (U.S. Pat. No. 5,730,979 the disclosure of which is hereby incorporated by reference).

Antibodies against CD122 (also called “interleukin-2 receptor subunit beta”, IL2RB) or its ligand IL-2 can also be used to deplete T-cells or NK-cells. CD122 is a subunit of the interleukin 2 receptor (IL2R), which is involved in T cell-mediated immune responses, and is present in 3 forms with respect to ability to bind interleukin 2. The low affinity form of IL2R is a monomer of the alpha subunit and is not involved in signal transduction. The intermediate affinity form consists of an alpha/beta subunit heterodimer, while the high affinity form consists of an alpha/beta/gamma subunit heterotrimer. Both the intermediate and high affinity forms of the receptor are involved in receptor-mediated endocytosis and transduction of mitogenic signals from interleukin 2. The use of alternative promoters results in multiple transcript variants encoding the same protein.

Antibodies against CD56 can be used for NK cell depletion. For example, IMGN901 is a CD56-targeting antibody-drug conjugate designed for selective delivery of the cytotoxic maytansinoid DM1 with a maximum tolerated dose (MTD) of about 75 mg/m² and which may be administered at doses of, for example, from about 1 to about 60 mg/m².

Immunotherapeutic agents that inhibit interaction of CD40 and CD40 ligand can also be used to deactivate endogenous T-cells in combination with an immunotherapeutic agent inhibiting CD47-SIRPα. CD40 is a costimulatory protein found on antigen presenting cells (APCs) and is required for their activation. These APCs include phagocytes (macrophages and dendritic cells) and B cells. CD40 is part of the TNF receptor family. The primary activating signaling molecules for CD40 are IFN1 and CD40 ligand (CD4OL). [72] “CD40 ligand” (“CD4OL”, also called “CD154”) is a type II transmembrane protein. CD4OL was originally considered restricted to activated T lymphocytes, functioning as a mediator of T cell-dependent B cell activation, proliferation, and differentiation. Expression of CD4OL plays a functional role as a central mediator of immunity and inflammation of the tumor necrosis factor (TNF) gene superfamily. CD40/CD4OL interaction is essential for the development of thymus-dependent humoral immune responses. CD4OL modulates physiologic processes, such as T cell-mediated effector functions and general immune responses required for appropriate host defense, but also triggers the expression of pro-inflammatory mediators, such as cytokines, adhesion molecules, and matrix degrading activities.

Ablation of T-cells or NK cells or both can be performed in further combination with one or more agents effective to deplete other cells of the immune system. For example, MCL1 apoptosis regulator, BCL2 family member (MCL1) inhibitors can be used to ablate NK cell. In various embodiments, an ablation regime as described herein, is combined with an inhibitor of MCL1 apoptosis regulator, BCL2 family member (MCL1, TM; EAT; MCL1L; MCL1S; Mcl-1; BCL2L3; MCL1-ES; bcl2-L-3; mcl1/EAT; NCBI Gene ID: 4170). Examples of MCL1 inhibitors include AMG-176, AMG-397, S-64315, AZD-5991, 483-LM, A-1210477, UMI-77, JKY-5-037, APG-3526 and those described in WO2018183418, WO2016033486, and WO2017147410.

III. Immunotherapeutic Agents Inhibiting CD47-SIRPα

Such agents include antibodies specifically binding to CD47 or SIRPα. Such agents also include a CD47 ECD fused to an Fc, which functions similarly to antibodies against SIRPα, or a SIRPα fused to an Fc, which functions similarly to antibodies against CD47. (See Zhang et al., Antibody Therapeutics, Volume 1, Issue 2, 21 Sep. 2018, Pages 27-32). Preferred antibodies antagonize CD47-SIRPα interaction without conferring an activating signal through either receptor.

CD47 is also known as any of IAP, MER6, and OA3. Human CD47, which is targeted by immunotherapeutic agents in treatment of humans, has been assigned exemplary accession numbers NCBI Gene ID:961 and UniProt Q08722.

Examples of suitable anti-CD47 antibodies include clones B6H12, 5F9, 8B6, C3, (for example as described in WO2011/143624) CC9002 (Vonderheide, Nat Med 2015; 21: 1122-3, 2015), and SRF231 (Surface Oncology). Suitable anti-CD47 antibodies include human, humanized or chimeric versions of such antibodies, antibodies binding to the same epitope or competing therewith for binding to CD47. Humanized antibodies (e.g., hu5F9-IgG4-WO2011/143624) are especially useful for in vivo applications in humans due to their low antigenicity. Similarly caninized, felinized antibodies and the like are especially useful for applications in dogs, cats, and other species respectively.

Some humanized antibodies specifically binds to human CD47 comprising a variable heavy (VH) region containing the VH complementarity regions, CDR1, CDR2 and CDR3, respectively set forth in SEQ ID NO: 20, 21 and 22 of WO2011/143624 (SEQ ID NOS:1-3 herein); and a variable light (VL) region containing the VL complementarity regions, CDR1, CDR2 and CDR3, respectively set forth in SEQ ID NO:23, 24 and 25 of WO2011/143624 (SEQ ID NOS:4-6 herein). Some humanized antibodies include a heavy chain variable region selected from SEQ ID NO: 36, SEQ ID NO: 37 and SEQ ID NO: 38 of WO2011/143624 (SEQ ID NOS:7-9 herein) and a light chain variable region selected from SEQ ID NO: 41, SEQ ID NO: 42 and SEQ ID NO: 43 of WO2011/143624 (SEQ ID NOS. 10-12 herein). Magrolimab, a humanized form of 5F9, is a preferred antibody.

Other examples of immunotherapeutic agents against CD47 inhibiting its interaction with SIRPα include anti-CD47 mAbs (Vx-1004), anti-human CD47 mAbs (CNTO-7108), CC-90002, CC-90002-ST-001, NI-1701, NI-1801, RCT-1938, ALX-148, RRx-001, DSP-107, VT-1021, TTI-621, TTI-622, IMM-02, and SGN-CD47M.

Additional examples of antibodies against CD47 are provided in e.g., WO199727873, WO199940940, WO2002092784, WO2005044857, WO2009046541, WO2010070047, WO2011143624, WO2012170250, WO2013109752, WO2013119714, WO2014087248, WO2015191861, WO2016022971, WO2016023040, WO2016024021, WO2016081423, WO2016109415, WO2016141328, WO2016188449, WO2017027422, WO2017049251, WO2017053423, WO2017121771, WO2017194634, WO2017196793, WO2017215585, WO2018075857, WO2018075960, WO2018089508, WO2018095428, WO2018137705, WO2018233575, WO2019027903, WO2019034895, WO2019042119, WO2019042285, WO2019042470, WO2019086573, WO2019108733, WO2019138367, WO2019144895, WO2019157843, WO2019179366, WO2019184912, WO2019185717, WO2019201236, WO2019238012, WO2019241732, WO2020019135, WO2020036977, WO2020043188 and WO2020009725.

Suitable anti-SIRPα antibodies specifically bind SIRPα (without activating/stimulating enough of a signaling response to inhibit phagocytosis) and inhibit an interaction between SIRPα and CD47. Human SIRPα, which is targeted by immunotherapeutic agents in treatment of humans, has been assigned exemplary accession numbers NCBI Gene ID: 140885; and UniProt P78324. Suitable anti-SIRPα antibodies include fully human, humanized or chimeric versions of such antibodies. Some exemplary anti-SIRPα antibodies defined by their Kabat CDRs and variable regions are provided in Table 1 below.

TABLE 1
anti-SIRPα antibodies

| SEQ ID | | |
|---|---|---|
| NO: | ID | Sequence |
| 13 | 1H9 CDR-H1 | SYWIT |
| 14 | 1H9 CDR-H2 | DIYPGSGSTNHIEKFKS |
| 15 | 1H9 CDR-H3 | GYGSSYGYFDY |
| 16 | 1H9 CDR-L1 | RASENIYSYLA |
| 17 | 1H9 CDR-L2 | TAKTLAE |
| 18 | 1H9 CDR-L3 | QHQYGPPFT |
| 19 | Humanized | QVQLVQSGAE VKKPGASVKV SCKASGYTFT SYWITWVKQA |
| | 1H9 V_{H} | PGQGLEWIGD IYPGSGSTNH IEKFKSKATL TVDTSISTAY MELSRLRSDD |
| | | TAVYYCATGY GSSYGYFDYW GQGTLVTVSS |
| 20 | Humanized | DIQMTQSPSS LSASVGDRVT ITCRASENIY SYLAWYQQKP GKAPKLLIYT |
| | 1H9 V_{L} | AKTLAEGVPS RFSGSGSGTD FTLTISSLQP EDFATYYCQH QYGPPFTFGQ |
| | | GTKLEIK |
| 21 | 3C2 CDR-H1 | SYWMH |
| 22 | 3C2 CDR-H2 | NIDPSDSDTHYNQKFKD |
| 23 | 3C2 CDR-H3 | GYSKYYAMDY |
| 24 | 3C2 CDR-L1 | RSSQSIVHSYGNTYLE |
| 25 | 3C2 CDR-L2 | KVSNRFS |
| 26 | 3C2 CDR-L3 | FQGSHVPYT |
| 27 | Humanized | QVQLVQSGAE VKKPGASVKV SCKASGYTFT SYWMHWVRQA |
| | 3C2 V_{H} | PGQGLEWMGN IDPSDSDTHY NQKFKDRVTM TRDTSTSTVY |
| | | MELSSLRSED TAVYYCARGY SKYYAMDYWG QGTLVTVSS |
| 28 | Humanized | DIVMTQTPLS LSVTPGQPAS ISCRSSQSIV HSYGNTYLEW YLQKPGQSPQ |
| | 3C2 V_{L} | LLIYKVSNRF SGVPDRFSGS GSGTDFTLKI SRVEAEDVGV YYCFQGSHVP |
| | | YTFGQGTKLE IK |
| 29 | Humanized | QVQLVQSGAEVKKPGASVKVSCKASGYTFTSYWITWVKQAPGQGLEWIGDI |
| | 1H9 HC | YPGSGSTNHIEKFKSKATLTVDTSISTAYMELSRLRSDDTAVYYCATGYGSSYG |
| | (full-length) | YFDYWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSN |
| | | TKVDKKVEPKSCDKTHTCPPCPAPELLGGPSVFLFPPKPKDTLMISRTPEVTCV |
| | | VVDVSHEDPEVKFNWYVDGVEVHNAKTKPREEQYASTYRVVSVLTVLHQD |
| | | WLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRW |
| | | QQGNVFSCSVMHEALHNHYTQKSLSLSPG |
| 30 | Humanized | DIQMTQSPSSLSASVGDRVTITCRASENIYSYLAWYQQKPGKAPKLLIYTAKTL |
| | 1H9 LC (full- | AEGVPSRFSGSGSGTDFTLTISSLQPEDFATYYCQHQYGPPFTFGQGTKLEIKR |
| | length) | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| 31 | Humanized | QVQLVQSGAEVKKPGASVKVSCKASGYTFTSYWMHWVRQAPGQGLEWM |
| | 3C2 HC (full- | GNIDPSDSDTHYNQKFKDRVTMTRDTSTSTVYMELSSLRSEDTAVYYCARGY |
| | length) | SKYYAMDYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYASTYRVVSVLTV |
| | | LHQDWLNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLYSKLTVD |
| | | KSRWQQGNVFSCSVMHEALHNHYTQKSLSLSPG |
| 32 | Humanized | DIVMTQTPLSLSVTPGQPASISCRSSQSIVHSYGNTYLEWYLQKPGQSPQLLIY |
| | 3C2 LC (full- | KVSNRFSGVPDRFSGSGSGTDFTLKISRVEAEDVGVYYCFQGSHVPYTFGQGT |
| | length) | KLEIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVTKSF |
| | | NRGEC |
| 33 | 9B11 CDR- | DYYIH |
| | H1 | |
| 34 | 9B11 CDR- | RIDPEDGETKYAPKFQG |
| | H2 | |
| 35 | 9B11 CDR- | GGFAY |
| | H3 | |
| 36 | 9B11 CDR- | ASSSVSSSYLY |
| | L1 | |
| 37 | 9B11 CDR- | STSNLAS |
| | L2 | |
| 38 | 9B11 CDR- | HQWSSHPYT |
| | L3 | |
| 39 | 9B11 V_{H} | EVQLQQSGAELVKPGASVKLSCTASGFNIKDYYIHWVKQRTEQGLEWIGRID |
| | | PEDGETKYAPKFQGKATITADTSSNTAYLQLNSLTSEDTAVYSCAKGGFAYW |
| | | GQGTLVTVSA |
| 40 | 9B11 V_{L} | QIVLTQSPAIMSASPGEKVTLTCSASSSVSSSYLYWYQQKPGSSPKLWIYSTSN |
| | | LASGVPARFSGSGSGTSYSLTISSMEAEDAASYFCHQWSSHPYTFGGGTKLEI |
| | | K |
| 41 | 7E11 CDR- | SYWMH |
| | H1 | |
| 42 | 7E11 CDR- | NIDPSDSDTHYNQKFKD |
| | H2 | |
| 43 | 7E11 CDR- | SYGNYGENAMDY |
| | H3 | |
| 44 | 7E11 CDR- | RSSQSIVHSYGNTYLE |
| | L1 | |
| 45 | 7E11 CDR- | KVSNRFS |
| | L2 | |
| 46 | 7E11 CDR- | FQGSHVPFT |
| | L3 | |
| 47 | 7E11 V_{H} | QVKLQESGAELVRPGSSVKLSCKASGYTFTSYWMHWVKQRPIQGLEWIGNI |
| | | DPSDSDTHYNQKFKDKATLTVDNSSSTAYMQLSSLTSEDSAVYYCASYGNYG |
| | | ENAMDYWGQGTSVTVSS |
| 48 | 7E11 V_{L} | DILMTQTPLSLPVSLGDQASISCRSSQSIVHSYGNTYLEWYLQKPGQSPKLLIYK |
| | | VSNRFSGVPDRFSGSGSGTDFTLKISRVEAEDLGVYYCFQGSHVPFTFGSGTK |
| | | LEIK |

An exemplary antibody from the above table is humanized 1H9 comprising a heavy chain variable region of SEQ ID NO:19 and light chain variable region of SEQ ID NO:20 and a human IgG1 constant region mutated for reduced effector function (N297A, EU numbering). Further exemplary antibodies are KWAR23 (Ring et al., Proc. Natl. Acad. Sci. USA. 2017 Dec. 5; 114(49): E10578-E10585, WO2015/138600), MY-1, Effi-DEM also known as B1765063 (Boehringer Ingelheim) (Zhang et al., Antibody Therapeutics, Volume 1, Issue 2, 21 Sep. 2018, Pages 27-32). Humanized antibodies are especially useful for in vivo applications in humans due to their low antigenicity. Similarly caninized, felinized, and so forth antibodies are especially useful for applications in dogs, cats, and other species respectively. Other examples of anti-SIRPα-antibodies include FSI-189 (Forty Seven, Inc.), ES-004, ADU1805 (Aduro Biotech and, Voets et al., J. Immunother. Cancer. 2019; 7: 340), and CC-95251 (Celgene, Uger & Johnson, Expert Opinion on Biological Therapy, 20:1, 5-8, DOI: 10.1080/14712598.2020.1685976).

Other examples of antibodies targeting SIRPα are provided in WO200140307, WO2002092784, WO2007133811, WO2009046541, WO2010083253, WO2011076781, WO2013056352, WO2015138600, WO2016179399, WO2016205042, WO2017178653, WO2018026600, WO2018057669, WO2018107058, WO2018190719, WO2018210793, WO2019023347, WO2019042470, WO2019175218, WO2019183266, WO2020013170 and WO2020068752.

Immunotherapeutic agents also include soluble CD47 polypeptides that specifically bind SIRPα and reduce the interaction between CD47 on a T-cell or NK cell and SIRPα on a phagocytic cell (see, e.g., WO2016179399). Such polypeptides can include the entire ECD or a portion thereof with the above functionality. A suitable soluble CD47 polypeptide specifically binds SIRPα without activating or stimulating signaling through SIRPα because activation of SIRPα would inhibit phagocytosis. Instead, suitable soluble CD47 polypeptides facilitate the phagocytosis of endogenous HCSPs. A soluble CD47 polypeptide can be fused to an Fc (e.g., as described in US20100239579).

Other examples of agents binding to SIRPα and inhibiting its interaction with CD47 are described in WO200140307, WO2002092784, WO2007133811, WO2009046541, WO2010083253, WO2011076781, WO2013056352, WO2015138600, WO2016179399, WO2016205042, WO2017178653, WO2018026600, WO2018057669, WO2018107058, WO2018190719, WO2018210793, WO2019023347, WO2019042470, WO2019175218, WO2019183266, WO2020013170 and WO2020068752.

Immunotherapeutic reagents also include soluble SIRPα polypeptides specifically binding to CD47 and inhibiting its interaction with SIRPα. Exemplary agents include ALX148 (Kauder et al., Blood 2017 130:112) and TTI-622 and TTI-661 Trillium). Such agents can include the entire SIRPα ECD or any portion thereof with the above functionality. The SIRPα reagent will usually comprise at least the d1 domain of SIRPα. The soluble SIRPα polypeptide can be fused to an Fc region. Exemplary SIRP α polypeptides termed “high affinity SIRPα reagent”, which includes SIRPα-derived polypeptides and analogs thereof (e.g., CV1-hlgG4, and CV1 monomer are described in WO2013/109752. High affinity SIRPα reagents are variants of the native SIRPα protein. The amino acid changes that provide for increased affinity are localized in the d1 domain, and thus high affinity SIRPα reagents comprise a d1 domain of human SIRPα, with at least one amino acid change relative to the wild-type sequence within the d1 domain. Such a high affinity SIRPα reagent optionally comprises additional amino acid sequences, for example antibody Fc sequences; portions of the wild-type human SIRPα protein other than the d1 domain, including without limitation residues 150 to 374 of the native protein or fragments thereof, usually fragments contiguous with the d1 domain; and the like. High affinity SIRPα reagents may be monomeric or multimeric, i.e. dimer, trimer, tetramer, and so forth. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more.

Immunotherapeutic agents directed at CD47 or SIRPα with an Fc region can have any of the human isotypes, e.g., IgG1, IgG2, IgG3 or IgG4. Human IgG4 or IgG2 isotype or IgG1 mutated to reduce effector functions can be used because effector functions are not required for inhibiting the CD47-SIRPα interaction.

IV. General Characteristics of Antibodies

The production of other non-human monoclonal antibodies, e.g., murine, guinea pig, primate, rabbit or rat, against an antigen can be accomplished by, for example, immunizing the animal with the antigen or a fragment thereof, or cells bearing the antigen. See Harlow & Lane, Antibodies, A Laboratory Manual (CSHP NY, 1988) (incorporated by reference for all purposes). Such an antigen can be obtained from a natural source, by peptide synthesis or by recombinant expression. Optionally, the antigen can be administered fused or otherwise complexed with a carrier protein. Optionally, the antigen can be administered with an adjuvant. Several types of adjuvant can be used as described below. Complete Freund's adjuvant followed by incomplete adjuvant is preferred for immunization of laboratory animals.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. Nos. 5,859,205 6,881,557, Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85, 90, 95 or 100% of corresponding residues defined by Kabat are identical.

Although humanized antibodies often incorporate all six CDRs (preferably as defined by Kabat) from a mouse antibody, they can also be made with less than all CDRs (e.g., at least 3, 4, or 5 CDRs from a mouse antibody) (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions.

A human antibody can be isolated from a human, or otherwise result from expression of human immunoglobulin genes (e.g., in a transgenic mouse, in vitro or by phage display). Methods for producing human antibodies include the trioma method of Oestberg et al., Hybridoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. Nos. 5,877,397, 5,874,299, 5,814,318, 5,789,650, 5,770,429, 5,661,016, 5,633,425, 5,625,126, 5,569,825, 5,545,806, Nature 148, 1547-1553 (1994), Nature Biotechnology 14, 826 (1996), Kucherlapati, WO 91/10741 (1991) and phage display methods (see, e.g. Dower et al., WO 91/17271 and McCafferty et al., WO 92/01047, U.S. Pat. Nos. 5,877,218, 5,871,907, 5,858,657, 5,837,242, 5,733,743 and 5,565,332.

Antibodies are screened for specific binding to their intended target. Antibodies may be further screened for binding to a specific region of the target (e.g., containing a desired epitope), competition with a reference antibody, agonism or antagonism of cells bearing the antigen. Non-human antibodies can be converted to chimeric, veneered or humanized forms as described above.

The choice of constant region depends, in part, whether antibody-dependent cell-mediated cytotoxicity, antibody dependent cellular phagocytosis and/or complement dependent cytotoxicity are desired. For example, human isotypes IgG1 and IgG3 have complement-dependent cytotoxicity and human isotypes IgG2 and IgG4 do not. Light chain constant regions can be lambda or kappa. Human IgG1 and IgG3 also induce stronger cell mediated effector functions than human IgG2 and IgG4.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of a one or more other isotypes. Reference to a human constant region includes a constant region with any natural allotype or any permutation of residues occupying polymorphic positions in natural allotypes.

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). Exemplary substitutions include a Gln at position 250 and/or a Leu at position 428, S or N at position 434, Y at position 252, T at position 254, and E at position 256. N434A (EU numbering). Increased FcRn binding is advantageous in making the hybrid proteins of the present invention compete more strongly with endogenous IgG for binding to FcRn. Also numerous mutations are known for reducing any of ADCC, ADP or CMC. (see, e.g., Winter et al., U.S. Pat. No. 5,624,821; Tso et al., U.S. Pat. No. 5,834,597; and Lazar et al., Proc. Natl. Acad. Sci. USA 103:4005, 2006). For example, substitution any of positions 234, 235, 236 and/or 237 reduce affinity for Fcγ receptors, particularly FcγRI receptor (see, e.g., U.S. Pat. No. 6,624,821). Optionally, positions 234, 236 and/or 237 in human IgG2 are substituted with alanine and position 235 with glutamine or glutamic acid. (See, e.g., U.S. Pat. No. 5,624,821.) Other substitutions reducing effector function include A at position 268, G or A at position 297, L at position 309, A at position 322, G at position 327, S at position 330, S at position 331, S at position 238, A at position 268, L at position 309. Some examples of mutations enhancing effector function include S239D, 1332E, A330L and combinations thereof.

As noted, in some embodiments, the Fc region of an antibody comprise one or more amino acid modifications that promote an increased serum half-life of the anti-binding molecule. Mutations that increase the half-life of an antibody have been described. In one embodiment, the Fc region or Fc domain of one or both of the CD3-targeting heavy chain and the HIV antigen-targeting heavy chain comprise a methionine to tyrosine substitution at position 252 (EU numbering), a serine to threonine substitution at position 254 (EU numbering), and a threonine to glutamic acid substitution at position 256 (EU numbering). See, e.g., U.S. Pat. No. 7,658,921. This type of mutant, designated as a “YTE mutant” exhibits a four-fold increased half-life relative to wild-type versions of the same antibody (Dall' Acqua, et al., J Biol Chem, 281: 23514-24 (2006); Robbie, et al., Antimicrob. Agents Chemotherap., 57(12):6147-6153 (2013)). In certain embodiments, the Fc region or Fc domain of one or both of the CD3-targeting heavy chain and the HIV antigen-targeting heavy chain comprise an IgG constant domain comprising one, two, three or more amino acid substitutions of amino acid residues at positions 251-257, 285-290, 308-314, 385-389, and 428-436 (EU numbering). Alternatively, M428L and N434S (“LS”) substitutions can increase the pharmacokinetic half-life of the multi-specific antigen binding molecule. In other embodiments, the Fc region comprises a M428L and N434S substitution (EU numbering). In other embodiments, the Fc region or Fc domain of one or both of the CD3-targeting heavy chain and the HIV antigen-targeting heavy chain comprise T250Q and M428L (EU numbering) mutations. In other embodiments, the Fc region comprise H433K and N434F (EU numbering) mutations.

As noted, the Fc region of an antibody can include post-translational and/or amino acid modifications that increase effector activity, e.g., have improved FcγIIIa binding and increased antibody-dependent cellular cytotoxicity (ADCC). In some embodiments, the Fc region or Fc domain of the antibody comprises DE modifications (i.e., S239D and I332E by EU numbering) in the Fc region. In some embodiments, the Fc region or Fc domain of the antibody comprises DEL modifications (i.e., S239D, I332E and A330L by EU numbering) in the Fc region. In some embodiments, the Fc region or Fc domain of the antibody comprises DEA modifications (i.e., S239D, I332E and G236A by EU numbering) in the Fc region. In some embodiments, the Fc region or Fc domain of the antibody comprises DEAL modifications (i.e., S239D, I332E, G236A and A330L by EU numbering) in the Fc region. See, e.g., U.S. Pat. Nos. 7,317,091; 7,662,925; 8,039,592; 8,093,357; 8,093,359; 8,383,109; 8,388,955; 8,735,545; 8,858,937; 8,937,158; 9,040,041; 9,353,187; 10,184,000; and 10,584,176. Additional amino acid modifications that increase effector activity, e.g., have improved FcγIIIa binding and increased antibody-dependent cellular cytotoxicity (ADCC) include without limitation (EU numbering) F243L/R292P/Y300L/V3051/P396L; S298A/E333A/K334A; or L234Y/L235Q/G236W/S239M/H268D/D270E/S298A on a first Fc domain and D270E/K326D/A330M/K334E on a second Fc domain. Amino acid mutations that increase C1q binding and complement-dependent cytotoxicity (CDC) include without limitation (EU numbering) S267E/H268F/S324T or K326W/E333S. Fc region mutations that enhance effector activity are reviewed in, e.g., Wang, et al., Protein Cell (2018) 9(1): 63-73; and Saunders, Front Immunol. (2019) 10:1296.

In other embodiments, the antibody or antigen-binding fragment thereof has modified glycosylation, which, e.g., may be introduced post-translationally or through genetic engineering. In some embodiments, the antibody or antigen-binding fragment thereof is afucosylated, e.g., at a glycosylation site present in the antibody or antigen-binding fragment thereof. Most approved monoclonal antibodies are of the IgG1 isotype, where two N-linked biantennary complex-type oligosaccharides are bound to the Fc region. The Fc region exercises the effector function of ADCC through its interaction with leukocyte receptors of the FcγR family. Afucosylated monoclonal antibodies are monoclonal antibodies engineered so that the oligosaccharides in the Fc region of the antibody do not have any fucose sugar units.

Antibodies of interest for ablation may be tested for their ability to induce ADCC. Antibody-associate ADCC activity can be monitored and quantified through detection of either the release of label or lactate dehydrogenase from the lysed cells, or detection of reduced target cell viability (e.g. annexin assay). Assays for apoptosis may be performed by terminal deoxynucleotidyl transferase-mediated digoxigenin-1 1-dUTP nick end labeling (TUNEL) assay (Lazebnik et al., Nature: 371, 346 (1994). Cytotoxicity may also be detected directly by detection kits, such as Cytotoxicity Detection Kit from Roche Applied Science (Indianapolis, Ind.). Antibodies can likewise be tested for their ability to induce antibody dependent phagocytosis (ADP) on for example AML LSC as described by WO/2009/091601.

In some embodiments, an immunotherapeutic agent is conjugated to an effector moiety. The effector moiety can be any number of molecules, including labeling moieties such as radioactive labels or fluorescent labels, or can be a cytotoxic moiety. Cytotoxic agents include cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin, saporin, auristatin-E and the like. Cytotoxic agents also include radiochemicals made by conjugating radioisotopes to antibodies. Targeting the cytotoxic moiety to transmembrane proteins serves to increase the local concentration of the cytotoxic moiety in the targeted area.

V. Engineered T-cells or NK-cells

T-cells or NK-cells to be engineered can be autologous or allogenic to the subject to be treated. Genetic engineering typically involves genetically modifying a T-cell or NK-cell to direct it to target T-cells bearing a target-associated antigen. A target cell can be, for example, cancer cell or pathogen-infected cells. One class of T-cell or NK modification is referred to as chimeric antigen receptor or CAR. This technology is referred to as CAR-T when applied to T-cells or CAR-NK when applied to NK-cells.

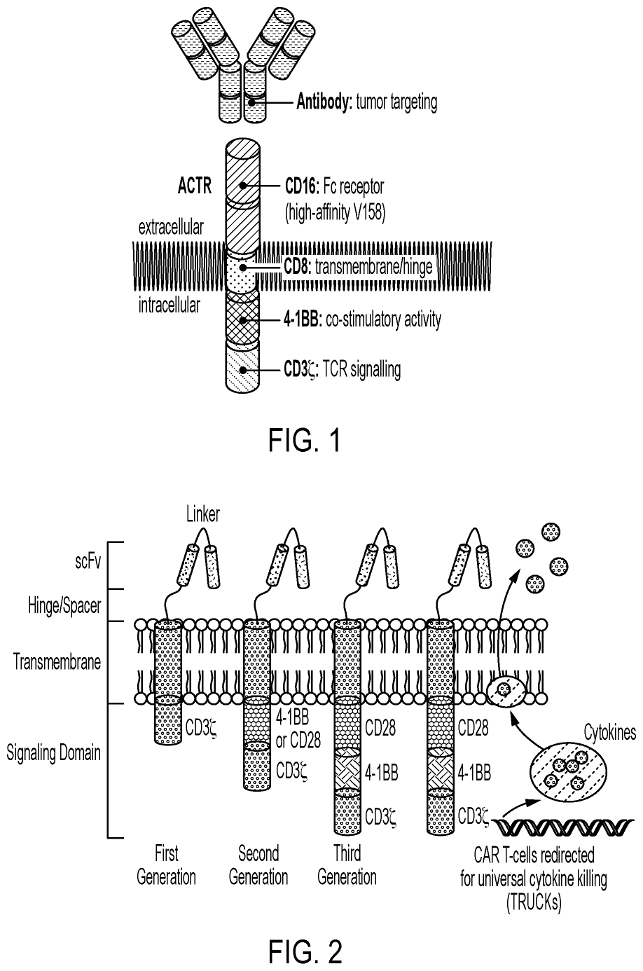

A CAR construct consists of three components: an extracellular component, a transmembrane domain and an intracellular signaling domain. The extracellular domain can be an antigen-recognition site, generally provided as an scFv derived from the variable regions of both the heavy and light chains of a monoclonal antibody, fused together via a flexible linker. Alternatively a Fab fragment can be used. Alternatively, the extracellular domain can be a universal adapter, such as an FcγR extracellular domain that provides a binding site for an antibody with an Fc region. The extracellular domain can also be streptavidin, which serves as a universal adapter for biotin linked antibodies. The extracellular domain can be connected via an antibody hinge region to a transmembrane domain. The hinge region imparts flexibility for adequate orientation and binding to the antigen. Different types of transmembrane domains can be used including a CD3-ζ chain of the T-cell receptor, CD4, CD8, or CD28, OX40, 4-1BB, Lck and/or ICOS. The transmembrane domain is linked at its intracellular end to an endodomain, which transmits activation signals to T-cells. “First-generation” CARs used a single intracellular signaling domain (CD3-ζ chain alone), whereas second- and third-generation CARs incorporate one or more additional costimulatory signaling domains, such as CD28, CD137, or OX40 to render them more potent. Fourth generate CAR, also known as TRUCKS, further engineer T-cells to express cytokines, which promote activation of the cells after introduction into a subject. Examples of CARs are shown in FIG. 1. The construction and use of CAR and CAR-T are reviewed by Sadelain et al. (Cancer Discov 3:388-98, 2013).

Alternatively, T-cells can be engineered to express alpha and beta T cell receptor chains (Ping et al., Protein Cell 9, 254-266 (2018)). Nucleic acids encoding these chains are isolated from single T-cell clones isolated from patient blood or tumor cells. These nucleic acids are introduced into a vector, such as a lentivirus or retrovirus, and introduced into T-cells, which are expanded in vitro and reintroduced into a patient.

CAR constructs can also be used to direct natural killer (NK) cell activity (Hermanson & Kaufman (2015, Front Immunol 6:195) and Carlsten & Childs (2015, Front Immunol 6:266)). Like T-cells, NK-cells can be transfected with CAR expression constructs and used to induce an immune response. Because NK-cells do not require HLA matching, they can be used as allogeneic effector cells (Harmanson & Kaufman, Front Immunol. 2015 Apr. 28; 6:195). Also, peripheral blood NK-cells (PB-NK), of use for therapy, can be isolated from donors by a simple blood draw. NK cells can also be harvested from umbilical cord blood, bone marrow, or embryonic stem cells (NK cells can be expanded in vitro with autologous or genetically modified allogenic feeder cells and/or stimulation with IL-2, IL-12, IL-15, IL-18. IL-21 and type I interferons (Becker et al. Cancer Immunol. Immunother. 65, 477-484 (2016)). The CAR constructs of use can contain similar elements to those used to make CAR-T-cells. CAR-NK-cells can contain a targeting molecule, such as a scFv or Fab, that binds to a disease associated antigen, such as a tumor-associated antigen (TAA), or to a hapten on a targetable construct. The cell-targeting scFv or Fab may be linked via a transmembrane domain to one or more intracellular signaling domains to effect lymphocyte activation. Signaling domains that can be incorporated into CAR-NK-cells include CD3-zeta, CD28, 4-1BB, DAP10 and OX40. As an alternative to drawing autologous or allogenic NK-cells from a subject, NK cell lines can be used including NK-92, NKG, YT, NK-YS, HANK-1, YTS and NKL cells. Transfection of such cell lines with genes encoding IL-2 and/or IL-15 can reduce dependence on the need for exogenous cytokines for in vivo persistence and cell population expansion.

Constructs to be introduced into T or NK-cells can be incorporated in an expression vector, such as a retroviral or lentiviral vector, for transfer into T-cells or NK-cells. Transposons and gene editing techniques, such as CRISPR-CAS9, zinc finger proteins or TALEN® transcriptional activators can also be used. Following infection, transfection, lipofection or alternative means of introducing the vector into the host cell, and expansion of the modified cells, the cells are administered to a subject to induce an immune response against target T-cells expressing an antigen recognized by the T-cells or NK-cells. Binding of CARs on the surface of transduced T-cells or NK-cells to antigens expressed by a target cell activates the T or NK cell. Activation of T or NK-cells by CARs does not require antigen processing and presentation by the HLA system.

A cancer-associated antigen is expressed at a significantly higher level at the protein level on cancerous cells than tissue matched normal cells or the subject or a control subject. Examples of cancer-associated that can be targeted by engineered T-cells include alpha-folate receptor (ovarian and epithelial cancers), CAIX (renal carcinoma), CD19 (B-cell malignancies, CLL, ALL), CD20 (B-cell malignancies, lymphomas), CD22 (B-cell malignancies), CD23 (CLL), CD24 (pancreatic CA), CD30 (lymphomas), CD33 (AML), CD38 (NHL), CD44v7/8 (cervical CA), CEA (colorectal CA), EGFRvIII (glioblastoma), EGP-2 (multiple malignancies), EGP-40 (colorectal CA), EphA2 (glioblastoma), Erb-B2 (breast, prostate, colon CA), FBP (ovarian CA), G.sub.D2 (neuroblastoma, melanoma), G.sub.D3 (melanoma), HER2 (pancreatic CA, ovarian CA, glioblastoma, osteosarcoma), HMW-MAA (melanoma), IL-11Rα (osteosarcoma), IL-13Rα2 (glioma, glioblastoma), KDR (tumor vasculature), kappa-light chain (B-cell malignancies), Lewis Y (various carcinomas), L1 (neuroblastoma), MAGE-A1 (melanoma), mesothelin (mesothelioma), MUC1 (breast and ovarian CA), MUC16 (ovarian CA), NKG2D (myeloma, ovarian CA), NY-ESO-1 (multiple myeloma), oncofetal antigen (various tumors), PSCA (prostate CA), PSMA (prostate CA), ROR1 (B-CLL), TAG-72 (adenocarcinomas), and VEGF-R2 (tumor neovasculature). (Sadelain et al., Cancer Discov 3:388-98, 2013). Other tumor-associated antigens that can be targeted include alpha-fetoprotein (AFP), alpha-actinin-4, A3, antigen specific for A33 antibody, ART-4, B7, Ba 733, BAGE, BrE3-antigen, CA125, CAMEL, CAP-1, carbonic anhydrase IX, CASP-8/m, CCLI9, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD126, CD132, CD133, CD138, CD147, CD154, CDC27, CDK-4/m, CDKN2A, CTLA4, CXCR4, CXCR7, CXCL12, HIF-1α, colon-specific antigen-p (CSAp), CEA (CEACAM-5), CEACAM-6, c-Met, DAM, EGFR, EGFRvIII, EGP-1 (TROP-2), EGP-2, ELF2-M, Ep-CAM, fibroblast growth factor (FGF), Flt-1, Flt-3, folate receptor, G250 antigen, GAGE, gp100, GRO-.beta., HLA-DR, HM1.24, human chorionic gonadotropin (HCG) and its subunits, HER2/neu, HMGB-1, hypoxia inducible factor (HIF-1), HSP70-2M, HST-2, IGF-1R, IFN-γ, IFN-, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5ac, MUC13, MUC16, MUM-1/2, MUM-3, NCA66, NCA95, NCA90, pancreatic cancer mucin, PD1 receptor, placental growth factor, p53, PLAGL2, prostatic acid phosphatase, PSA, PRAME, PSMA, PIGF, ILGF, ILGF-R, IL-6, IL-25, RS5, RANTES, T101, SAGE, S100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, an oncogene marker or an oncogene product (see, e.g., Sensi et al., Clin Cancer Res 2006, 12:5023-32; Parmiani et al., J Immunol 2007, 178:1975-79; Novellino et al. Cancer Immunol Immunother. 2005, 54:187-207).

Exemplary antibodies against tumor associated antigens include to, hA19 (anti-CD19, U.S. Pat. No. 7,109,304), hR1 (anti-IGF-1R, U.S. Pat. No. 8,883,162), hPAM4 (anti-MUC5ac, U.S. Pat. No. 7,282,567), hA20 (anti-CD20, U.S. Pat. No. 7,151,164), hIMMU31 (anti-AFP, U.S. Pat. No. 7,300,655), hLL1 (anti-CD74, U.S. Pat. No. 7,312,318), hLL2 (anti-CD22, U.S. Pat. No. 5,789,554), hMu-9 (anti-CSAp, U.S. Pat. No. 7,387,773), hL243 (anti-HLA-DR, U.S. Pat. No. 7,612,180), hMN-14 (anti-CEACAM-5, U.S. Pat. No. 6,676,924), hMN-15 (anti-CEACAM-6, U.S. Pat. No. 8,287,865), hRS7 (anti-EGP-1, U.S. Pat. No. 7,238,785), hMN-3 (anti-CEACAM-6, U.S. Pat. No. 7,541,440), hRFB4 (anti-CD22, U.S. Pat. No. 9,139,649), Ab124 and Ab125 (anti-CXCR4, U.S. Pat. No. 7,138,496).

Engineered T-cells can also be used to treat subjects infected with pathogenic organisms, such as bacteria, viruses or fungi. Exemplary fungi that may be treated include Microsporum, Trichophyton, Epidermophyton, Sporothrix schenckii, Cryptococcus neoformans, Coccidioides immitis, Histoplasma capsulatum, Blastomyces dermatitidis or Candida albican. Exemplary viruses include human immunodeficiency virus (HIV), herpes virus, cytomegalovirus, rabies virus, influenza virus, human papilloma virus, hepatitis B virus, hepatitis C virus, Sendai virus, feline leukemia virus, Reo virus, polio virus, human serum parvo-like virus, simian virus 40, respiratory syncytial virus, mouse mammary tumor virus, Varicella-Zoster virus, dengue virus, rubella virus, measles virus, adenovirus, human T-cell leukemia viruses, Epstein-Barr virus, murine leukemia virus, mumps virus, vesicular stomatitis virus, Sindbis virus, lymphocytic choriomeningitis virus or blue tongue virus. Exemplary bacteria include Bacillus anthracis, Streptococcus agalactiae, Legionella pneumophilia, Streptococcus pyogenes, Escherichia coli, Neisseria gonorrhoeae, Neisseria meningitidis, Pneumococcus spp., Hemophilis influenzae B, Treponema pallidum, Lyme disease spirochetes, Pseudomonas aeruginosa, Mycobacterium leprae, Brucella abortus, Mycobacterium tuberculosis or a Mycoplasma. Exemplary use of ADCs against infectious agents are disclosed in Johannson et al. (AIDS 20:1911-15, 2006) and Chang et al., PLos One 7:e41235, 2012).

Examples of antibodies against pathogens include P4D10 (anti-HIV), CR6261 (anti-influenza), exbivirumab (anti-hepatitis B), felvizumab (anti-respiratory syncytial virus), foravirumab (anti-rabies virus), motavizumab (anti-respiratory syncytial virus), palivizumab (anti-respiratory syncytial virus), panobacumab (anti-Pseudomonas), rafivirumab (anti-rabies virus), regavirumab (anti-cytomegalovirus), sevirumab (anti-cytomegalovirus), tivirumab (anti-hepatitis B), and urtoxazumab (anti-E. coli).

T-cells or NK-cells can also be genetically engineered to have a mutant version of a receptor against which an immunotherapeutic agent is directed in a depleting regime. The mutant version binds to the immunotherapeutic agent with reduced affinity if at all compared with the wildtype version thus providing the engineered T-cells or NK-cells a selective advantage over endogenous T-cells or NK-cells in the continued or residual presence of the immunotherapeutic agent from the depleting regime. The mutation can be present in one or more amino acid positions of the receptor forming the epitope bound by such an immunotherapeutic agent. For example, if the depletion regime involves an antibody against CD3 binding to an epitope X, then T-cells can be genetically manipulated to express CD3 with a mutation in epitope X such that the immunotherapeutic agent does not bind or binds only to a reduced extent to the mutated CD3. Alternatively, the mutation can reduce or eliminate immunotherapeutic agent binding allosterically. Such a mutation preferably does not significantly reduce binding of the receptor to its ligand or co-receptor. Additionally or alternatively, T-cells or NK-cells can be genetically engineered to express CD47 mutated such that an immunotherapeutic agent against CD47 used in a depleting regime does not bind or binds at only a reduced extent to CD47. Alternatively, the mutation can reduce or eliminate binding of an immunotherapeutic agent against CD47 allosterically. Such a mutation preferably does not significantly reduce binding of CD47 to SIRPα. Genetic engineering to introduce mutations can be performed using gene editing tools, such as zinc fingers, TALEN® transcriptional activators or CRISPR/CAS9.

Prior to expansion and genetic modification of the T-cells of the invention, a source of T-cells can be obtained from a subject (see, e.g., U.S. Pat. No. 9,783,591; Levin et al., Mol. Therapy Methods & Clinical Development 4, 92-101 (2017)). T-cells can be obtained from sources such as peripheral blood mononuclear cells, bone marrow, lymph node tissue, cord blood, thymus tissue, tissue from a site of infection, ascites, pleural effusion, spleen tissue, and tumors. For example, cells from the circulating blood of an individual are obtained by apheresis. The apheresis product typically contains lymphocytes, including T-cells, monocytes, granulocytes, B cells, other nucleated white blood cells, red blood cells, and platelets. The cells collected by apheresis can be washed to remove the plasma fraction and to place the cells in an appropriate buffer or media for subsequent processing steps. After washing, the cells can be resuspended in a variety of biocompatible buffers, such as, for example, Ca.sup.2+-free, Mg.sup.2+-free PBS, PlasmaLyte A, or other saline solution with or without buffer. T-cells can be separated from other lymphocytes. T-cells can be used unfractionated in subsequent steps or a specific subpopulation of T-cells, such as CD3+, CD28+, CD4+, CD8+, CD45RA+, and CD45RO+ T-cells, can be further isolated by positive or negative selection techniques. For example, T-cells can be isolated by incubation with anti-CD3/anti-CD28 conjugated beads, such as DYNABEADS® M-450 CD3/CD28 T. Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD₄+ cells by negative selection, a monoclonal antibody cocktail typically includes antibodies to CD14, CD20, CD11b, CD16, HLA-DR, and CD8.

Before or after genetic modification or both, T-cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and US20060121005. T-cells can be expanded by contact with an anti-CD3 antibody together with feeder cells or cytokines, such as IL-2. Alternatively, T-cell can be expanded by contact with beads coated with anti-CD3 and andt-CD28 antibodies, as described above. A combination of artificial antigen presenting cells and IL-2 can also be used. Culture conditions can be further refined to polarized T-cells to a specific phenotype, e.g., Th2 or Th17 during expansion (Levine et al., Molecular Therapy: Methods & Clinical Development 4, 92-101 (2017)).

Replacement T cells, NK cells or other cellular therapies can be administered with one or more agent to promoter growth of T cells, NK cells or other cells. For example, replacement T cells or other cellular therapies can be combined with administration an agonist of fms-related receptor tyrosine kinase 3 (FLT3); FLK2; STK1; CD135; FLK-2; NCBI Gene ID: 2322). Examples of FLT3 agonists include CDX-301 and GS-3583. Replacement T cells, NK cells or other cellular therapies can also be combined with an inhibitor of cytokine inducible SH2 containing protein (CISH; CIS; G18; SOCS; CIS-1; BACTS2; NCBI Gene ID: 1154). Examples of CISH inhibitors include those described in WO2017100861, WO2018075664 and WO2019213610.

As previously discussed, the methods can include administering immune cells engineered to express chimeric antigen receptors (CARs) or T cell receptors (TCRs) TCRs. In particular embodiments, a population of immune cells is engineered to express a CAR, wherein the CAR comprises a cancer antigen-binding domain. In other embodiments, a population of immune cells is engineered to express T cell receptors (TCRs) engineered to target tumor derived peptides presented on the surface of tumor cells. In one embodiment, the immune cell engineered to express chimeric antigen receptors (CARs) or T cell receptors (TCRs) TCRs is a T cell. In another embodiment, the immune cell engineered to express chimeric antigen receptors (CARs) or T cell receptors (TCRs) TCRs is an NK cell.

With respect to the structure of a CAR, in some embodiments, the CAR comprises an antigen binding domain, a transmembrane domain, and an intracellular signaling domain. In some embodiments, the intracellular domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCERIG), FcR beta (Fc Epsilon RIb), CD79a, CD79b, Fcgamma RIIa, DAP10, and DAP12 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8a, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

In some embodiments, the costimulatory domain comprises a functional domain of one or more proteins selected from the group consisting of CD27, CD28, 4-1BB(CD137), OX40, CD30, CD40, PD-1, ICOS, CD2, CD7, LIGHT, NKG2C, lymphocyte function-associated antigen-1 (LFA-1), MYD88, B7-H3, a ligand that specifically binds with CD83, CDS, ICAM-1, GITR, BAFFR, HVEM (LIGHTR), SLAMF7, NKp80 (KLRFI), CD19, CD4, CD8a, CD8beta, IL2R beta, IL2R gamma, IL7R alpha, ITGA4, VLA1, CD49a, ITGA4, IA4, CD49D, ITGA6, VLA-6, CD49f, ITGAD, ITGAE, CD103, ITGAL, CD1A (NCBI Gene ID: 909), CD1B (NCBI Gene ID: 910), CD1C (NCBI Gene ID: 911), CD1D (NCBI Gene ID: 912), CD1E (NCBI Gene ID: 913), ITGAM, ITGAX, ITGB1, CD29, ITGB2 (CD18, LFA-1), ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments, the transmembrane domain comprises a transmembrane domain derived from a protein selected from the group consisting of the alpha, beta or zeta chain of the T-cell receptor, CD28, CD3 epsilon, CD3 delta, CD3 gamma, CD45, CD4, CD5, CD7, CD8a, CD8 beta, CD9, CD11a, CD11b, CD11c, CD11d, CD16, CD18, CD22, CD33, CD37, CD64, CD80, CD86, CD134, CD137, CD154, KIRDS2, OX40, CD2, CD27, ICOS (CD278), 4-1BB(CD137), GITR, CD40, BAFFR, HVEM (LIGHTR), SLAMF7, NKp80 (KLRF1), CD19, CD19a, IL2R beta, IL2R gamma, IL7R alpha, ITGA1, VLA1, CD49a, ITGA4, IA4, CD49D, ITGA6, VLA-6, CD49f, ITGAD, CD1A, CD1B, CD1C, CD1D, CD1E, ITGAE, CD103, ITGAL, ITGAM, ITGAX, ITGB1, ITGB2, ITGB7, CD29, ITGB2 (LFA-1, CD18), ITGB7, TNFR2, DNAM1 (CD226), SLAMF4 (CD244, 2B4), CD84, CD96 (TACTILE), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C activating NK cell receptors, an Immunoglobulin protein, BTLA, CD247, CD276 (B7-H3), CD30, CD84, CDS, cytokine receptor, Fc gamma receptor, GADS, ICAM-1, Ig alpha (CD79a), integrins, LAT, a ligand that binds with CD83, LIGHT, MHC class 1 molecule, PAG/Cbp, TNFSF14, a Toll ligand receptor, TRANCE/RANKL, or a fragment, truncation, or a combination thereof.

In some embodiments, the CAR comprises a hinge domain. A hinge domain may be derived from a protein selected from the group consisting of the CD2, CD3 delta, CD3 epsilon, CD3 gamma, CD4, CD7, CD8a, CD8.beta., CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, IgG1, IgG2, IgG3, IgG4, IgA, IgD, IgE, IgM or fragment or combination thereof.

In some embodiments, the TCR or CAR antigen binding domain or the immunotherapeutic agent described herein (e.g., monospecific or multi-specific antibody or antigen-binding fragment thereof or antibody mimetic) binds a tumor-associated antigen (TAA). In some embodiments, the tumor-associated antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRvIII); ganglioside G2 (GD2); ganglioside GD3 (αNeuSAc(2-8)αNeuSAc(2-3)βDGaip(1-4)bDGIcp(1-1)Cer); ganglioside GM3 (αNeuSAc(2-3)βDGalp(1-4)βDGIcp(1-1)Cer); GM-CSF receptor; TNF receptor superfamily member 17 (TNFRSF17, BCMA); B-lymphocyte cell adhesion molecule; Tn antigen ((Tn Ag) or (GaINAcu-Ser/Thr)); prostate-specific membrane antigen (PSMA); Receptor tyrosine kinase-like orphan receptor 1 (RORI); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Rα2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); HLA class I antigen A-2 alpha; HLA antigen; Lewis(Y)antigen; CD24; Platelet-derived growth factor receptor beta (PDGFR-beta); Stage-specific embryonic antigen-4 (SSEA-4); CD20; delta like 3 (DLL3); Folate receptor alpha; Folate receptor beta, GDNF alpha 4 receptor, Receptor tyrosine-protein kinase, ERBB2 (Her2/neu); Mucin 1, cell surface associated (MUC1); APRIL receptor; ADP ribosyl cyclase-1; Ephb4 tyrosine kinase receptor, DCAMKL1 serine threonine kinase, Aspartate beta-hydroxylase, epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); ephrin type-A receptor 3 (EphA3), Fucosyl GM1; sialyl Lewis adhesion molecule (sLe); transglutaminase 5 (TGS5); high molecular weight-melanomaassociatedantigen (HMWMAA); o-acetyl-GD2 ganglioside (OAcGD2); Folate receptor beta; tumor endothelial marker 1 (TEM1/CD248); tumor endothelial marker 7-related (TEM7R); six transmembrane epithelial antigen of the prostate I (STEAP1); claudin 6 (CLDN6); thyroid stimulating hormone receptor (TSHR); G protein-coupled receptor class C group 5, member D (GPRCSD); IL-15 receptor (IL-15); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (ORS IE2); TCR Gamma Alternate Reading Frame Protein (TARP); Wilms tumor protein (WT1); Cancer/testis antigen 1 (NY-ESO-1); Cancer/testis antigen 2 (LAGE-Ia); Melanoma associated antigen 1 (MAGE-A1); Melanoma associated antigen 3 (MAGE-A3); Melanoma associated antigen 4 (MAGE-A4); T cell receptor beta 2 chain C; ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MADCT-1); melanoma cancer testis antigen-2 (MAD-CT-2); Fos-related antigen 1; tumor protein p53, (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin-A1; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1(CYP IBI); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES I); lymphocyte-specific protein tyrosine kinase (LCK); A kinase anchor protein 4 (AKAP-4); Peptidoglycan recognition protein, synovial sarcoma, X breakpoint 2 (SSX2); Receptor for Advanced Glycation End products (RAGE-1); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); Glypican-2 (GPC2); Glypican-3 (GPC3); Fc receptor-like 5 (FCRL5); and immunoglobulin lambda-like polypeptide 1 (IGLL1). In some embodiments, the target is an epitope of the tumor associated antigen presented in an MHC.

In some embodiments, the cancer antigen is selected from CD150, 5T4, ActRIIA, B7, TNF receptor superfamily member 17 (TNFRSF17, BCMA), CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, HER1-HER2 in combination, HER2-HER3 in combination, HERV-K, HIV-1 envelope glycoprotein gp120, HIV-1 envelope glycoprotein gp41, HLA-DR, HLA class I antigen alpha G, HM1.24, K-Ras GTPase, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, LI-CAM, MAGE A3, MAGE-A1, MART-1, MUC1, NKG2C ligands, NKG2D Ligands, NYESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-I, a G-protein coupled receptor, alpha-fetoprotein (AFP), an angiogenesis factor, an exogenous cognate binding molecule (ExoCBM), oncogene product, anti-folate receptor, c-Met, carcinoembryonic antigen (CEA), cyclin (D 1), ephrinB2, epithelial tumor antigen, estrogen receptor, fetal acetylcholine e receptor, folate binding protein, gp100, hepatitis B surface antigen, Epstein-Barr nuclear antigen 1, Latent membrane protein 1, Secreted protein BARF1, P2X7 purinoceptor, Syndecan-1, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, P2-Microgiobuiin, Fc Receptor-like 5 (FcRL5).

Examples of cell therapies include without limitation: AMG-119, Algenpantucel-L, ALOFISEL®, Sipuleucel-T, (BPX-501) rivogenlecleucel U.S. Pat. No. 9,089,520, WO2016100236, AU-105, ACTR-087, activated allogeneic natural killer cells CNDO-109-AANK, MG-4101, AU-101, BPX-601, FATE-NK100, LFU-835 hematopoietic stem cells, Imilecleucel-T, baltaleucel-T, PNK-007, UCARTCS1, ET-1504, ET-1501, ET-1502, ET-190, CD19-ARTEMIS, ProHema, FT-1050-treated bone marrow stem cell therapy, CD4CARNK-92 cells, SNK-01, NEXI-001, CryoStim, AlloStim, lentiviral transduced huCART-meso cells, CART-22 cells, EGFRt/19-28z/4-1BBL CAR T cells, autologous 4H11-28z/fIL-12/EFGRt T cell, CCR5-SBC-728-HSPC, CAR4-1BBZ, CH-296, dnTGFbRII-NY-ESOc259T, Ad-RTS-IL-12, IMA-101, IMA-201, CARMA-0508, TT-18, CMD-501, CMD-503, CMD-504, CMD-502, CMD-601, CMD-602, CSG-005, LAAP T-cell therapy, PD-1 knockout T cell therapy (esophageal cancer/NSCLC), anti-MUC1 CAR T-cell therapy (esophageal cancer/NSCLC), anti-MUC1 CAR T-cell therapy+PD-1 knockout T cell therapy (esophageal cancer/NSCLC), anti-KRAS G12D mTCR PBL, anti-CD123 CAR T-cell therapy, anti-mutated neoantigen TCR T-cell therapy, tumor lysate/MUC1/survivin PepTivator-loaded dendritic cell vaccine, autologous dendritic cell vaccine (metastatic malignant melanoma, intradermal/intravenous), anti-LeY-scFv-CD28-zeta CAR T-cells, PRGN-3005, iC9-GD2-CAR-IL-15 T-cells, HSC-100, ATL-DC-101, MIDRIX4-LUNG, MIDRIXNEO, FCR-001, PLX stem cell therapy, MDR-101, GeniusVac-Mel4, ilixadencel, allogeneic mesenchymal stem cell therapy, romyelocel L, CYNK-001, ProTrans, ECT-100, MSCTRAIL, dilanubicel, FT-516, ASTVAC-2, E-CEL UVEC, CK-0801, allogenic alpha/beta CD3+ T cell and CD19+ B cell depleted stem cells (hematologic diseases, TBX-1400, HLCN-061, umbilical cord derived Hu-PHEC cells (hematological malignancies/aplastic anemia), AP-011, apceth-201, apceth-301, SENTI-101, stem cell therapy (pancreatic cancer), ICOVIR15-cBiTE, CD33HSC/CD33 CAR-T, PLX-Immune, SUBCUVAX, CRISPR allogeneic gamma-delta T-cell based gene therapy (cancer), ex vivo CRISPR allogeneic healthy donor NK-cell based gene therapy (cancer), ex-vivo allogeneic induced pluripotent stem cell-derived NK-cell based gene therapy (solid tumor), and anti-CD20 CAR T-cell therapy (non-Hodgkin's lymphoma).

VI. Co-Therapies for Treating Cancer

Cellular therapies can be combined with one or more second agents or modalities effective to treat cancer. Such an agent or modality can be administered before, during or after depletion of T-cell or NK-cells. As used herein, the term “chemotherapeutic agent” or “chemotherapeutic” (or “chemotherapy” in the case of treatment with a chemotherapeutic agent) is meant to encompass any non-proteinaceous (e.g., non-peptidic) chemical compound useful in the treatment of cancer. Examples of chemotherapeutic agents include but not limited to: alkylating agents such as thiotepa and cyclophosphamide (CYTOXAN®); alkyl sulfonates such as busulfan, improsulfan, and piposulfan; aziridines such as benzodepa, carboquone, meturedepa, and uredepa; ethylenimines and methylamelamines including altretamine, triethylenemelamine, triethylenephosphoramide, triethylenethiophosphoramide, and trimemylolomelamine; acetogenins, e.g., bullatacin and bullatacinone; a camptothecin, including synthetic analog topotecan; bryostatin, callystatin; CC-1065, including its adozelesin, carzelesin, and bizelesin synthetic analogs; cryptophycins, particularly cryptophycin 1 and cryptophycin 8; dolastatin; duocarmycin, including the synthetic analogs KW-2189 and CBI-TMI; eleutherobin; 5-azacytidine; pancratistatin; a sarcodictyin; spongistatin; nitrogen mustards such as chlorambucil, chlornaphazine, cyclophosphamide, glufosfamide, evofosfamide, bendamustine, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammall and calicheamicin phil1), dynemicin including dynemicin A, bisphosphonates such as clodronate, an esperamicin, neocarzinostatin chromophore and related chromoprotein enediyne antibiotic chromophores, aclacinomycins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as demopterin, methotrexate, pteropterin, and trimetrexate; purine analogs such as cladribine, pentostatin, fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenishers such as frolinic acid; radiotherapeutic agents such as Radium-223, 177-Lu-PSMA-617; trichothecenes, especially T-2 toxin, verracurin A, roridin A, and anguidine; taxoids such as paclitaxel (TAXOL®), abraxane, docetaxel (TAXOTERE®), cabazitaxel, BIND-014, tesetaxel; platinum analogs such as cisplatin and carboplatin, NC-6004 nanoplatin; aceglatone; aldophosphamide glycoside; aminolevulinic acid; eniluracil; amsacrine; hestrabucil; bisantrene; edatraxate; defofamine; demecolcine; diaziquone; elformithine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; trabectedin, triaziquone; 2,2′,2″-trichlorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; NUC-1031; FOLFOX (folinic acid, 5-fluorouracil, oxaliplatin); FOLFIRI (folinic acid, 5-fluorouracil, irinotecan); FOLFOXIRI (folinic acid, 5-fluorouracil, oxaliplatin, irinotecan), FOLFIRINOX (folinic acid, 5-fluorouracil, irinotecan, oxaliplatin), and pharmaceutically acceptable salts, acids, or derivatives of any of the above. Such agents can be conjugated onto an antibody or any targeting agent described herein to create an antibody-drug conjugate (ADC) or targeted drug conjugate.

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors.

Examples of anti-estrogens and SERMs include, for example, tamoxifen (including NOLVADEX™), raloxifene, droloxifene, 4-hydroxytamoxifen, trioxifene, keoxifene, LY117018, onapristone, and toremifene (FARESTON®).

Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®).

Examples of anti-androgens include apalutamide, abiraterone, enzalutamide, flutamide, galeterone, nilutamide, bicalutamide, leuprolide, goserelin, ODM-201, APC-100, ODM-204.

Additional examples of agents for targeting cancers include: alpha-fetoprotein modulators, such as ET-1402, and AFP-TCR; Anthrax toxin receptor 1 modulator, such as anti-TEM8 CAR T-cell therapy; TNF receptor superfamily member 17 (TNFRSF17, BCMA), such as bb-2121 (ide-cel), bb-21217, JCARH125, UCART-BCMA, ET-140, MCM-998, LCAR-B38M, CART-BCMA, SEA-BCMA, BB212, ET-140, P-BCMA-101, AUTO-2 (APRIL-CAR), JNJ-68284528; nti-CLL-1 antibodies, (see, for example, WO/2017/173384); Anti-PD-L1-CAR tank cell therapy, such as KD-045; Anti-PD-L1 t-haNK, such as PD-L1 t-haNK; anti-CD45 antibodies, such as 131I-BC8 (lomab-B); anti-HER3 antibodies, such as ␣M716, GSK2849330; anti-CD52 antibodies, such as alemtuzumab; APRIL receptor modulator, such as anti-BCMA CAR T-cell therapy, Descartes-011; ADP ribosyl cyclase-1/APRIL receptor modulator, such as dual anti-BCMA/anti-CD38 CAR T-cell therapy; CART-ddBCMA; B7 homolog 6, such as CAR-NKp30 and CAR-B7H6; B-lymphocyte antigen CD19, such as TBI-1501, CTL-119 huCART-19 T cells, liso-cel, JCAR-015 U.S. Pat. No. 7,446,190, JCAR-014, JCAR-017, (WO2016196388, WO2016033570, WO2015157386), axicabtagene ciloleucel (KTE-C19, Yescarta®), KTE-X19, U.S. Pat. Nos. 7,741,465, 6,319,494, UCART-19, EBV-CTL, T tisagenlecleucel-T (CTL019), WO2012079000, WO2017049166, CD19CAR-CD28-CD3zeta-EGFRt-expressing T cells, CD19/4-1BBL armored CAR T cell therapy, C-CAR-011, CIK-CAR.CD19, CD19CAR-28-zeta T cells, PCAR-019, MatchCART, DSCAR-01, IM19 CAR-T, TC-110; anti-CD19 CAR T-cell therapy (B-cell acute lymphoblastic leukemia, Universiti Kebangsaan Malaysia); anti-CD19 CAR T-cell therapy (acute lymphoblastic leukemia/Non-Hodgkin's lymphoma, University Hospital Heidelberg), anti-CD19 CAR T-cell therapy (silenced IL-6 expression, cancer, Shanghai Unicar-Therapy Bio-medicine Technology), MB-CART2019.1 (CD19/CD20), GC-197 (CD19/CD7), CLIC-1901, ET-019003, anti-CD19-STAR-T cells, AVA-001, BCMA-CD19 cCAR (CD19/APRIL), ICG-134, ICG-132 (CD19/CD20), CTA-101, WZTL-002, dual anti-CD19/anti-CD20 CAR T-cells (chronic lymphocytic leukemia/B-cell lymphomas), HY-001, ET-019002, YTB-323, GC-012 (CD19/APRIL), GC-022 (CD19/CD22), CD19CAR-CD28-CD3zeta-EGFRt-expressing Tn/mem; UCAR-011, ICTCAR-014, GC-007F, PTG-01, CC-97540; Allogeneic anti-CD19 CART cells, such as GC-007G; APRIL receptor modulator; SLAM family member 7 modulator, BCMA-CS1 cCAR; Autologous dendritic cell tumor antigen (ADCTA), such as ADCTA-SSI-G; B-lymphocyte antigen CD20, such as ACTR707 ATTCK-20, PBCAR-20A; Allogenic T cells expressing CD20 CAR, such as LB-1905; B-lymphocyte antigen CD19/B-lymphocyte antigen 22, such as TC-310; B-lymphocyte antigen 22 cell adhesion, such as UCART-22, JCAR-018 WO2016090190; NY-ESO-1 modulators, such as GSK-3377794, TBI-1301, GSK3537142; Carbonic anhydrase, such as DC-Ad-GMCAIX; Caspase 9 suicide gene, such as CaspaCIDe DLI, BPX-501; CCR5, such as SB-728; CCR5 gene inhibitor/TAT gene/TRIM5 gene stimulator, such as lentivirus vector CCR5 shRNA/TRIM5alpha/TAR decoy-transduced autologous CD34-positive hematopoietic progenitor cells; CDw123, such as MB-102, IM-23, JEZ-567, UCART-123; CD4, such as ICG-122; CD5 modulators, such as CD5.28z CART cells; Anti-CD22, such as anti-CD22 CART; Anti-CD30, such as TT-11; CD33, such as CIK-CAR.CD33, CD33CART; Dual anti-CD33/anti-CLL1, such as LB-1910; CD38, such as T-007, UCART-38; CD40 ligand, such as BPX-201, MEDI5083; CD56, such as allogeneic CD56-positive CD3-negative natural killer cells (myeloid malignancies); CD19/CD7 modulator, such as GC-197; T-cell antigen CD7 modulator, such as anti-CD7 CAR T-cell therapy (CD7-positive hematological malignancies); CD123 modulator, such as UniCAR02-T-CD123; Anti-CD276, such as anti-CD276 CART; CEACAM protein 5 modulators, such as MG7-CART; Claudin 6, such as CSG-002; Claudin 18.2, such as LB-1904; chlorotoxin, such as CLTX-CART; EBV targeted, such as CMD-003; MUC16EGFR, such as autologous 4H11-28z/fIL-12/EFGRt T cell; Endonuclease, such as PGN-514, PGN-201; Epstein-Barr virus specific T-lymphocytes, such as TT-10; Epstein-Barr nuclear antigen 1/Latent membrane protein 1/Secreted protein BARF1 modulator, such as TT-10×; Erbb2, such as CST-102, CIDeCAR; Ganglioside (GD2), such as 4SCAR-GD2; Gamma delta T cells, such as ICS-200; folate hydrolase 1 (FOLH1, Glutamate carboxypeptidase II, PSMA; NCBI Gene ID: 2346), such as CIK-CAR.PSMA, CART-PSMA-TGFβRDN, P-PSMA-101; Glypican-3(GPC3), such as TT-16, GLYCAR; hemoglobin, such as PGN-236; Hepatocyte growth factor receptor, such as anti-cMet RNA CAR T; HLA class I antigen A-2 alpha modulator, such as FH-MCVA2TCR; HLA class I antigen A-2 alpha/Melanoma associated antigen 4 modulator, such as ADP-A2M4CD8; HLA antigen modulator, such as FIT-001, NeoTCR-P1; Human papillomavirus E7 protein, such as KITE-439 (see, for example, WO/2015/184228); ICAM-1 modulator, such as AIC-100; Immunoglobulin gamma Fc receptor III, such as ACTR087; IL-12, such as DC-RTS-IL-12; IL-12 agonist/mucin 16, such as JCAR-020; IL-13 alpha 2, such as MB-101; IL-15 receptor agonist, such as PRGN-3006, ALT-803; interleukin-15/Fc fusion protein (e.g., XmAb24306); recombinant interleukin-15 (e.g., AM0015, NIZ-985); pegylated IL-15 (e.g., NKTR-255); IL-2, such as CST-101; Interferon alpha ligand, such as autologous tumor cell vaccine+systemic CpG-B+IFN-alpha (cancer); K-Ras GTPase, such as anti-KRAS G12V mTCR cell therapy; Neural cell adhesion molecule L1 L1CAM (CD171), such as JCAR-023; Latent membrane protein 1/Latent membrane protein 2, such as Ad5f35-LMPd1-2-transduced autologous dendritic cells; MART-1 melanoma antigen modulator, such as MART-1 F5 TCR engineered PBMC; Melanoma associated antigen 10, such as MAGE-A10C796T MAGE-A10 TCR; Melanoma associated antigen 3/Melanoma associated antigen 6 (MAGE A3/A6) such as KITE-718 (see, for example, WO/2014/043441); Mesothelin, such as CSG-MESO, TC-210; Mucin 1 modulator, such as ICTCAR-052, Tn MUC-1 CAR-T, ICTCAR-053; Anti-MICA/MICB, such as CYAD-02; NKG2D, such as NKR-2; Ntrkr1 tyrosine kinase receptor, such as JCAR-024; PRAMET cell receptor, such as BPX-701; Prostate stem cell antigen modulator, such as MB-105; Roundabout homolog 1 modulator, such as ATCG-427; Peptidoglycan recognition protein modulator, such as Tag-7 gene modified autologous tumor cell vaccine; PSMA, such as PSMA-CAR T-cell therapy (lentiviral vector, castrate-resistant prostate cancer); SLAM family member 7 modulator, such as IC9-Luc90-CD828Z; TGF beta receptor modulator, such as DNR.NPC T-cells; T-lymphocyte, such as TT-12; T-lymphocyte stimulator, such as ATL-001; TSH receptor modulator, such as ICTCAR-051; Tumor infiltrating lymphocytes, such as LN-144, LN-145; and Wilms tumor protein, such as JTCR-016, WT1-CTL, or ASP-7517. An example progesterone receptor antagonist includes onapristone.

In various embodiments, an agent for treating cancer as described above, can be combined with an anti-angiogenic agent. Anti-angiogenic agents that can be co-administered include, but are not limited to, retinoid acid and derivatives thereof, 2-methoxyestradiol, ANGIOSTATIN®, ENDOSTATIN®, regorafenib, necuparanib, suramin, squalamine, tissue inhibitor of metalloproteinase-1, tissue inhibitor of metalloproteinase-2, plasminogen activator inhibitor-1, plasminogen activator inhibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as I-azetidine-2-carboxylic acid (LACA), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, metalloproteinase inhibitors such as BB-94, inhibitors of S100A9 such as tasquinimod. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

In various embodiments, an agent for treating cancer as described above, is combined with an anti-fibrotic agent. Anti-fibrotic agents that can be co-administered include, but are not limited to, the compounds such as beta-aminopropionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. Nos. 5,021,456, 5,059,714, 5,120,764, 5,182,297, 5,252,608 relating to 2-(1-naphthyloxymemyl)-3-fluoroallylamine, and US 2004-0248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.

Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

Some chemotherapy agents are suitable for treating lymphoma or leukemia. These agents include aldesleukin, alvocidib, amifostine trihydrate, aminocamptothecin, antineoplaston A10, antineoplaston AS2-1, anti-thymocyte globulin, arsenic trioxide, Bcl-2 family protein inhibitor ABT-263, beta alethine, BMS-345541bortezomib (VELCADE®, PS-341), bryostatin 1, bulsulfan, campath-1H, carboplatin, carfilzomib (Kyprolis®), carmustine, caspofungin acetate, CC-5103, chlorambucil, CHOP (cyclophosphamide, doxorubicin, vincristine, and prednisone), cisplatin, cladribine, clofarabine, curcumin, CVP (cyclophosphamide, vincristine, and prednisone), cyclophosphamide, cyclosporine, cytarabine, denileukin diftitox, dexamethasone, docetaxel, dolastatin 10, doxorubicin, doxorubicin hydrochloride, DT-PACE (dexamethasone, thalidomide, cisplatin, doxorubicin, cyclophosphamide, and etoposide), enzastaurin, epoetin alfa, etoposide, everolimus (RAD001), FCM (fludarabine, cyclophosphamide, and mitoxantrone), FCR (fludarabine, cyclophosphamide, and rituximab), fenretinide, filgrastim, flavopiridol, fludarabine, FR (fludarabine and rituximab), geldanamycin (17 AAG), hyperCVAD (hyperfractionated cyclophosphamide, vincristine, doxorubicin, dexamethasone, methotrexate, and cytarabine), ICE (iphosphamide, carboplatin, and etoposide), ifosfamide, irinotecan hydrochloride, interferon alpha-2b, ixabepilone, lenalidomide (REVLIMID®, CC-5013), pomalidomide (POMALYST®/IMNOVID®)lymphokine-activated killer cells, MCP (mitoxantrone, chlorambucil, and prednisolone), melphalan, mesna, methotrexate, mitoxantrone hydrochloride, motexafin gadolinium, mycophenolate mofetil, nelarabine, obatoclax (GX15-070), oblimersen, octreotide acetate, omega-3 fatty acids, Omr-IgG-am (WNIG, Omrix), oxaliplatin, paclitaxel, palbociclib (PD0332991), pegfilgrastim, PEGylated liposomal doxorubicin hydrochloride, perifosin, prednisolone, prednisone, recombinant flt3 ligand, recombinant human thrombopoietin, recombinant interferon alfa, recombinant interleukin-11, recombinant interleukin-12, rituximab, R-CHOP (rituximab and CHOP), R-CVP (rituximab and CVP), R-FCM (rituximab and FCM), R-ICE (rituximab and ICE), and R MCP (rituximab and MCP), R-roscovitine (seliciclib, CYC202), sargramostim, sildenafil citrate, simvastatin, sirolimus, styryl sulphones, tacrolimus, tanespimycin, temsirolimus (CCI-779), thalidomide, therapeutic allogeneic lymphocytes, thiotepa, tipifarnib, vincristine, vincristine sulfate, vinorelbine ditartrate, SAHA (suberanilohydroxamic acid, or suberoyl, anilide, and hydroxamic acid), vemurafenib (Zelboraf®), venetoclax (ABT-199).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab (BEXXAR®), yttrium-90 ibritumomab tiuxetan (ZEVALIN®), and BEXXAR© with CHOP.

Treatment of non-Hodgkin's lymphomas (NHL), especially those of B cell origin, includes using monoclonal antibodies, standard chemotherapy approaches (e.g., CHOP (cyclophosphamide, doxorubicin, vincristine, and prednisone), CVP (cyclophosphamide, vincristine, and prednisone), FCM (fludarabine, cyclophosphamide, and mitoxantrone), MCP (Mitoxantrone, Chlorambucil, Prednisolone), all optionally including rituximab (R) and the like), radioimmunotherapy, and combinations thereof, especially integration of an antibody therapy with chemotherapy.

Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74.

Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab.

Examples of standard regimens of chemotherapy for NHL/B-cell cancers include CHOP, FCM, CVP, MCP, R-CHOP (rituximab, cyclophosphamide, doxorubicin, vincristine, and prednisone), R-FCM, R-CVP, and R MCP.

Examples of radioimmunotherapy for NHL/B-cell cancers include yttrium-90 ibritumomab tiuxetan (ZEVALIN®) and iodine-131 tositumomab (BEXXAR®).

Therapeutic treatments for mantle cell lymphoma (MCL) include combination chemotherapies such as CHOP, hyperCVAD, and FCM. These regimens can also be supplemented with the monoclonal antibody rituximab to form combination therapies R-CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE© or PS-341), or administering anti-angiogenesis agents such as thalidomide, especially in combination with rituximab.

Another treatment approach is administering drugs that lead to the degradation of Bcl-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are sirolimus, temsirolimus (TORISEL®, CCI-779), CC-115, CC-223, SF-1126, PQR-309 (bimiralisib), voxtalisib, GSK-2126458, and temsirolimus in combination with RITUXAN®, VELCADE®, or other chemotherapeutic agents.

Other recent therapies for MCL have been disclosed. Such examples include flavopiridol, palbociclib (PD0332991), R-roscovitine (seliciclib, CYC202), styryl sulphones, obatoclax (GX15-070), TRAIL, Anti-TRAIL death receptors DR4 and DR5 antibodies, temsirolimus (TORISEL©, CCI-779), everolimus (RAD001), BMS-345541, curcumin, SAHA, thalidomide, lenalidomide (REVLIMID®, CC-5013), and geldanamycin (17 AAG).

Therapeutic agents used to treat Waldenstrom's Macroglobulinemia (WM) include aldesleukin, alemtuzumab, alvocidib, amifostine trihydrate, aminocamptothecin, antineoplaston A10, antineoplaston AS2-1, anti-thymocyte globulin, arsenic trioxide, autologous human tumor-derived HSPPC-96, Bcl-2 family protein inhibitor ABT-263, beta alethine, bortezomib (VELCADE®), bryostatin 1, busulfan, campath-1H, carboplatin, carmustine, caspofungin acetate, CC-5103, cisplatin, clofarabine, cyclophosphamide, cyclosporine, cytarabine, denileukin diftitox, dexamethasone, docetaxel, dolastatin 10, doxorubicin hydrochloride, DT-PACE, enzastaurin, epoetin alfa, epratuzumab (hLL2-anti-CD22 humanized antibody), etoposide, everolimus, fenretinide, filgrastim, fludarabine, ibrutinib, ifosfamide, indium-111 monoclonal antibody MN-14, iodine-131 tositumomab, irinotecan hydrochloride, ixabepilone, lymphokine-activated killer cells, melphalan, mesna, methotrexate, mitoxantrone hydrochloride, monoclonal antibody CD19 (such as tisagenlecleucel-T, CART-19, CTL-019), monoclonal antibody CD20, motexafin gadolinium, mycophenolate mofetil, nelarabine, oblimersen, octreotide acetate, omega-3 fatty acids, oxaliplatin, paclitaxel, pegfilgrastim, PEGylated liposomal doxorubicin hydrochloride, pentostatin, perifosine, prednisone, recombinant flt3 ligand, recombinant human thrombopoietin, recombinant interferon alfa, recombinant interleukin-11, recombinant interleukin-12, rituximab, sargramostim, sildenafil citrate (VIAGRA®), simvastatin, sirolimus, tacrolimus, tanespimycin, thalidomide, therapeutic allogeneic lymphocytes, thiotepa, tipifarnib, tositumomab, ulocuplumab, veltuzumab, vincristine sulfate, vinorelbine ditartrate, vorinostat, WT1 126-134 peptide vaccine, WT-1 analog peptide vaccine, yttrium-90 ibritumomab tiuxetan, yttrium-90 humanized epratuzumab, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

Therapeutic agents used to treat diffuse large B-cell lymphoma (DLBCL) include cyclophosphamide, doxorubicin, vincristine, prednisone, anti-CD20 monoclonal antibodies, etoposide, bleomycin, many of the agents listed for WM, and any combination thereof, such as ICE and R ICE.

Examples of therapeutic agents used to treat chronic lymphocytic leukemia (CLL) include chlorambucil, cyclophosphamide, fludarabine, pentostatin, cladribine, doxorubicin, vincristine, prednisone, prednisolone, alemtuzumab, many of the agents listed for WM, and combination chemotherapy and chemoimmunotherapy, including the following common combination regimens: CVP, R-CVP, ICE, R-ICE, FCR, and FR.

Myelofibrosis inhibiting agents include, but are not limited to, hedgehog inhibitors, histone deacetylase (HDAC) inhibitors, and tyrosine kinase inhibitors. Non-limiting examples of hedgehog inhibitors are saridegib and vismodegib. Examples of HDAC inhibitors include, but are not limited to, pracinostat and panobinostat. Non-limiting examples of tyrosine kinase inhibitors are lestaurtinib, bosutinib, imatinib, radotinib, and cabozantinib.

Gemcitabine, nab-paclitaxel, and gemcitabine/nab-paclitaxel may be used with a JAK inhibitor and/or PI3Kδ inhibitor to treat hyperproliferative disorders.

Therapeutic agents used to treat bladder cancer include atezolizumab, carboplatin, cisplatin, docetaxel, doxorubicin, fluorouracil (5-FU), gemcitabine, idosfamide, Interferon alfa-2b, methotrexate, mitomycin, nab-paclitaxel, paclitaxel, pemetrexed, thiotepa, vinblastine, and any combination thereof.

Therapeutic agents used to treat breast cancer include albumin-bound paclitaxel, anastrozole, capecitabine, carboplatin, cisplatin, cyclophosphamide, docetaxel, doxorubicin, epirubicin, everolimus, exemestane, fluorouracil, fulvestrant, gemcitabine, Ixabepilone, lapatinib, Letrozole, methotrexate, mitoxantrone, paclitaxel, pegylated liposomal doxorubicin, pertuzumab, tamoxifen, toremifene, trastuzumab, vinorelbine, and any combinations thereof.

Therapeutic agents used to treat triple negative breast cancer include cyclophosphamide, docetaxel, doxorubicin, epirubicin, fluorouracil, paclitaxel, and combinations thereof.

Therapeutic agents used to treat colorectal cancer include bevacizumab, capecitabine, cetuximab, fluorouracil, irinotecan, leucovorin, oxaliplatin, panitumumab, ziv-aflibercept, and any combinations thereof.

Therapeutic agents used to treat castration-resistant prostate cancer include abiraterone, cabazitaxel, docetaxel, enzalutamide, prednisone, sipuleucel-T, and any combinations thereof.

Therapeutic agents used to treat esophageal and esophagogastric junction cancer include capecitabine, carboplatin, cisplatin, docetaxel, epirubicin, fluoropyrimidine, fluorouracil, irinotecan, leucovorin, oxaliplatin, paclitaxel, ramucirumab, trastuzumab, and any combinations thereof.

Therapeutic agents used to treat gastric cancer include capecitabine, carboplatin, cisplatin, docetaxel, epirubicin, fluoropyrimidine, fluorouracil, Irinotecan, leucovorin, mitomycin, oxaliplatin, paclitaxel, ramucirumab, trastuzumab, and any combinations thereof.

Therapeutic agents used to treat head & neck cancer include afatinib, bleomycin, capecitabine, carboplatin, cetuximab, cisplatin, docetaxel, fluorouracil, gemcitabine, hydroxyurea, methotrexate, nivolumab, paclitaxel, pembrolizumab, vinorelbine, and any combinations thereof.

Therapeutic agents used to treat hepatobiliary cancer include capecitabine, cisplatin, fluoropyrimidine, 5-fluorourcil, gemcitabine, oxaliplatin, sorafenib, and any combinations thereof.

Therapeutic agents used to treat hepatocellular carcinoma include capecitabine, doxorubicin, gemcitabine, sorafenib, and any combinations thereof.

Therapeutic agents used to treat non-small cell lung cancer (NSCLC) include afatinib, albumin-bound paclitaxel, alectinib, bevacizumab, bevacizumab biosimilar, cabozantinib, carboplatin, cisplatin, crizotinib, dabrafenib, docetaxel, erlotinib, etoposide, gemcitabine, nivolumab, paclitaxel, pembrolizumab, pemetrexed, ramucirumab, trametinib, trastuzumab, vandetanib, vemurafenib, vinblastine, vinorelbine, and any combinations thereof.

Therapeutic agents used to treat small cell lung cancer (SCLC) include bendamustine, carboplatin, cisplatin, cyclophosphamide, docetaxel, doxorubicin, etoposide, gemcitabine, ipilimumab, irinotecan, nivolumab, paclitaxel, temozolomide, topotecan, vincristine, vinorelbine, and any combinations thereof.

Therapeutic agents used to treat melanoma cancer include albumin bound paclitaxel, carboplatin, cisplatin, cobimetinib, dabrafenib, dacarbazine, IL-2, imatinib, interferon alfa-2b, ipilimumab, nitrosourea, nivolumab, paclitaxel, pembrolizumab, pilimumab, temozolomide, trametinib, vemurafenib, vinblastine, and any combinations thereof.

Therapeutic agents used to treat ovarian cancer include 5-flourouracil, albumin bound paclitaxel, altretamine, anastrozole, bevacizumab, capecitabine, carboplatin, cisplatin, cyclophosphamide, docetaxel, doxorubicin, etoposide, exemestane, gemcitabine, ifosfamide, irinotecan, letrozole, leuprolide acetate, liposomal doxorubicin, megestrol acetate, melphalan, olaparib, oxaliplatin, paclitaxel, Pazopanib, pemetrexed, tamoxifen, topotecan, vinorelbine, and any combinations thereof.

Therapeutic agents used to treat pancreatic cancer include 5-fluorourcil, albumin-bound paclitaxel, capecitabine, cisplatin, docetaxel, erlotinib, fluoropyrimidine, gemcitabine, irinotecan, leucovorin, oxaliplatin, paclitaxel, and any combinations thereof.

Therapeutic agents used to treat renal cell carcinoma include axitinib, bevacizumab, cabozantinib, erlotinib, everolimus, lenvatinib, nivolumab, pazopanib, sorafenib, sunitinib, temsirolimus, and any combinations thereof.

VII. Treatment Regimes

Immunotherapeutic agents, including antibodies and Fc fusion proteins, are administered to a subject in need thereof in a regime effective to achieve the desired purpose of reducing or eliminating endogenous T-cells or NK-cells. An effective regime refers to a combination of dose, frequency of administration and route of administration. Subjects in need include those having or at risk of cancer or pathogenic infection.

Immunotherapy agents inhibiting CD47-SIRPα are administered in a regime effective to promote reduction of T-cells or NK-cells or both by an immunotherapeutic agent against the T-cells or NK-cells, or both or combination of such agents (e.g., one immunotherapeutic agent against T-cells and one against NK-cells). Exemplary doses for immunotherapy agents inhibiting CD47-SIRPα are at least any of 0.05 mg/kg, 0.1 mg/kg, 0.5 mg/kg, 1 mg/kg up to any of 5 mg/kg, 10 mg/kg, 20 mg/kg, 30 mg/kg 40 mg/kg or 50 mg/kg including all combinations of lower and upper doses. Some exemplary ranges are 0.05 mg/kg-50 mg/kg, 0.1 mg/kg to 20 mg/kg, 1 mg/kg to 10 mg/kg or 10-30 mg/kg. Optionally, such immunotherapeutic agents, particularly those against CD47, can be administered initially at one or more priming doses, followed by one or more therapeutic doses (higher than the priming dose) to reduce undesired crosslinking of red blood cells, as described by e.g., WO2017181033. A preferred regime is a priming dose at 0.5 to 5 mg/kg, e.g., 1 mg/kg following by a therapeutic dose of 10-30 or 15-20 mg/kg. The therapeutic dose is administered e.g., 3-15 or 5-10 or 7 days after the priming dose. An immunotherapeutic agent against SIRPα is sometimes administered as a single dose and sometimes as two or more doses.

Exemplary doses of the agent depleting T-cells or NK-cells depend on the individual and the specific agent. Exemplary doses can be at least 50 ag/kg body weight, at least 250 ag/kg, at least 500 ag/kg, at least 750 ag/kg, at least 1 mg/kg, and up to 2.5 mg/kg, up to 5 mg/kg, up to 7.5 mg/kg, up to 10 mg/kg, up to 15 mg/kg, up to 25 mg/kg, up to 50 mg/kg, or up to 100 mg/kg including all combinations of lower and upper doses.

Combination or co-administration treatment with one or more immunotherapeutic agent against T cell or NK-cells and an immunotherapeutic agent inhibiting CD47 SIRPα involves administering the respective agents sufficiently proximal in time for the latter to promote reduction of T-cells or NK-cells by the former. Typically in combination regimes both (or all) agents are present at detectable levels in subject serum at the same time. In some combination regimes, a priming dose of an immunotherapeutic agent inhibiting CD47-SIRPα is administered followed by administration of a dose of immunotherapeutic agent against T-cells or NK-cells and a therapeutic dose of the immunotherapeutic agent inhibiting CD47-SIRPα at the same time. In some such regimes, the two or more agents are administered simultaneously by co-infusion.

Replacement T-cells or NK-cells, or both, can be administered after administration of the combined regime of immunotherapeutic agents specifically binding to T-cells or NK-cells or both and inhibiting CD47-SIRPα. The replacement T-cells can be administered either alone, or in combination with other components such as IL-2 or other cytokines. Optionally, replacement T-cells or NK-cells are administered 5-15 days after administration of the last does of immunotherapeutic agent against T-cells or NK-cells or the last dose of immunotherapeutic agent antagonizing CD47-SIRPα, if administered later. Thus, some regimes administer one or more dosages of immunotherapeutic agent antagonizing CD47-SIRPα, one or more dosage of immunotherapeutic agent against T-cells or NK-cells within a period of about 30 days, followed by administration of replacement T-cells or NK-cells 5-15 days after the last dose of the immunotherapeutic agent antagonizing CD47-SIRPα or immunotherapeutic agent against T-cells or NK-cells. Some regimes administer a priming dose of immunotherapeutic agent against CD47 followed 5-15 days later by a therapeutic dose of immunotherapeutic agent against CD47 and a dose of immunotherapeutic agent against T-cells or NK-cells on the same day, followed 5-15 days later by administration of replacement T-cells or NK-cells.

Optionally levels of endogenous T-cells or NK-cells and/or levels of immunotherapeutic agent against T-cells or NK-cells and/or levels of the immunotherapeutic agent inhibiting CD47-SIRPα are measured and replacement T-cells or NK-cells are administered when the level of endogenous T-cells or NK-cells fall below a threshold % of pretreatment levels (e.g., <50% or <5% or 25-75%) and levels of immunotherapeutic agent against T-cells and/or the immunotherapeutic agent inhibiting CD47-SIRPα fall below 25, 10, 5, or 1% of maximum levels or reach undetectable level.

Immunotherapeutic agents are typically administered as pharmaceutical compositions in which the agent is combined with one or more pharmaceutically acceptable carriers. A variety of aqueous carriers can be used, e.g., buffered saline and the like. These solutions are sterile and generally free of undesirable matter, and suitable for use in humans. These compositions may be sterilized by conventional techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and is selected primarily based on fluid volumes, viscosities, body weight and the like in accordance with the particular mode of administration selected and the patient's needs (e.g., Remington's Pharmaceutical Science (15th ed. 1980) and Goodman & Gillman, The Pharmacological Basis of Therapeutics (Hardman et al., eds., 1996)).

Immunotherapeutic agents are administered to subjects in need thereof. Some such subjects have a cancer, which can be a hemopoietic malignancy or solid tumor. Examples of such malignancies include multiple myeloma, Non-Hodgkin lymphoma, Hodgkin disease, acute myeloid leukemia, acute lymphoid leukemia, acute lymphoblastic leukemia, chronic myeloid leukemia; chronic lymphocytic leukemia, myeloproliferative disorders, Solid tumors include those of breast, prostate, brain, lung, kidney, liver, stomach, intestine, ovary, melanoma, and pancreas among others. Cancers particularly amenable to treatment include leukemias, lymphomas, myelomas and myelodysplastic syndrome. Other subjects in need have a pathogenic infection.

VIII. Regimes for Administering Engineered T-Cells or NK Cells

Engineered T-cells or NK-cells are administered parenterally, typically by intravenous infusion. Intratumoral intracranial, intraperitoneal, hepatic artery, or transcatheter arterial infusion can also be used. The dose of cells administered can depend on the desired purity of the infused cell composition, and the source of the cells. Exemplary dosages of cells for reintroduction are at least 10⁶, 10⁷, 10⁸, 10⁹, 10¹⁰, 10¹¹, or 10¹² engineered cells per patient, e.g., 10⁶-10¹², 10⁷-10⁹ or 5×10⁷-5×10⁸ engineered cells per patient. Because the engineered cells may not be 100% pure of unmodified cells, the total number of cells administered may be higher. The dose can be given in one infusion or split into two or more infusions within a period of about a week. Sometimes the infusion is split with dose escalation covering up to two log steps (or more).

IX. Monitoring

After introduction of genetically engineered T-cells or NK-cells into a subject, the ratio of engineered to total T-cells or NK-cells can be monitored. Engineered cells can be distinguished from endogenous by e.g., a nucleic acid hybridization assay or immunoassays. If the engineered cells are allogenic, there are many genetic differences a between the replacement and endogenous cells that can form the basis of a differential probe binding assay and sometimes differences in receptors that allow an immunoassay. If the engineered cells are autologous, the genetic modification of the engineered cells can distinguish them from endogenous cells by either a nucleic acid hybridization assay or immunoassay. The proportion of engineered to total T-cells or NK-cells may increase with time after introduction, for example so the proportion exceeds 30, 50, 75, 90 or 95% after six months.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the disclosure can be used in combination with any other unless specifically indicated otherwise. Although the present disclosure has been described in some detail by way of illustration and example for purposes of clarity and understanding, it will be apparent that certain changes and modifications may be practiced within the scope of the appended claims.

What is claimed is:

1. A method of performing T-cell or NK cell therapy in a subject in need thereof, comprising:
administering to the subject a combination therapy comprising an immunotherapeutic agent antagonizing CD47 interaction with SIRPα and an immunotherapeutic agent binding to a T-cell or NK cell antigen, thereby depleting endogenous T-cells or NK-cells of the subject, wherein the subject is also administered genetically engineered T-cells or NK-cells.

2. The method of claim 1, wherein the subject is administered the genetically engineered T-cells.

3. The method of claim 2, wherein the T-cell are genetically engineered to have a chimeric antigen receptor.

4. The method of claim 3, wherein the chimeric antigen receptor, comprises an scFv or Fab, a transmembrane domain and an intracellular signaling domain.

5. The method of claim 3, wherein the chimeric antigen receptor comprises a CD16 extracellular domain, a transmembrane domain and an intracellular signaling domain, wherein the CD16 domain is complexed with an Fc domain of an antibody.

6. The method of claim 2, wherein the genetically engineered T-cells are genetically engineered to express alpha and beta domains of a T-cell receptor.

7. The method of claim 1, wherein the genetically engineered NK-cells are administered.

8. The method of any preceding claim, wherein the immunotherapeutic agent antagonizing CD47 interaction with SIRPα is an antibody specifically binding to CD47.

9. The method of claim 8, wherein the antibody is magrolimab.

10. The method of claim 8 or 9, wherein the antibody specifically binding to CD47 is administered at a priming dose followed by a higher therapeutic dose.

11. The method of any one of claims 1-8, wherein the immunotherapeutic agent antagonizing CD47 interaction with SIRPα is an antibody specifically binding to SIRPα.

12. The method of claim 11, wherein the antibody comprises a heavy chain variable region having a sequence comprising SEQ ID NO:19 and a light chain variable region having a sequence comprising SEQ ID NO:20.

13. The method of claim 11, wherein the antibody specifically binding to SIRPα is any of FSI-189, ES-004, BI765063, ADU1805, and CC-95251.

14. The method of any preceding claim, wherein the immunotherapeutic agent antagonizing CD47 interaction with SIRPα is administered at a dose of 10-30 mg/kg.

15. The method of any one of claims 11-14, wherein a single dose of the antibody specifically biding to SIRPα is administered.

16. The method of any one of claims 11-14, wherein two or more doses of the antibody specifically binding to SIRPα are administered.

17. The method of any preceding claim, wherein the immunotherapeutic agent specifically binding to a T-cell antigen specifically binds to CD2, CD3, CD4, CD8, CD52, CD45 or ATG.

18. The method of any preceding claim, wherein the T-cells administered to the subject are autologous T-cells.

19. The method of any one of claims 1-17, wherein the T-cells administered to the subject are allogenic T-cells.

20. The method of any one of claims 1-17, wherein the T-cells administered to the subject have a T-cell receptor linked to an antibody against a cancer-associated antigen.

21. The method of any preceding claim, wherein the subject has a cancer expressing a cancer-associated antigen and the T-cells or NK-cells are engineered to bind to the antigen.

22. The method of any preceding claim, wherein the combination therapy is performed before the subject is administered the T-cells or NK-cells.

23. The method of any preceding claim, wherein the T-cells or NK cells administered to the subject are engineered for reduced binding to the immunotherapeutic agent specifically binding to the T-cell or NK cell antigen and/or the immunotherapeutic agent antagonizing CD47 interaction with SIRPα.

24. The method of any preceding claim, wherein the combination therapy does not include an antibody specifically binding to c-kit.

25. The method of any preceding claim, wherein the combination therapy does not include a genotoxic or myeloablative agent.

26. The method of any preceding claim, wherein the combination therapy does not include dimethyl busulfan.

27. The method of any preceding claim, wherein the subject has a cancer.

28. The method of claim 27, wherein the cancer is a leukemia, lymphoma, myeloma or myelodysplastic syndrome.

29. The method of claim 27, wherein the cancer is a hematological cancer.

30. The method of claim 27, wherein the cancer is a solid tumor.

31. The method of any one of claims 27-30, further comprising administering a second agent to treat the cancer.

32. The method of claim 30, wherein the subject is administered the second agent the before or during depletion of the T-cells or NK cells.

33. The method of claim 31 or 32, wherein the agent is a chemotherapeutic agent, anti-angiogenic agent, anti-fibrotic agent or monoclonal antibody against a cancer antigen.

34. The method of any preceding claim further comprising administering a flt3 agonist or CISH inhibitor after depletion of the T-cells or NK-cells to promote growth of the engineered T-cells or NK cells.

35. The method of any preceding claim further comprising administering an MCL1 inhibitor with the immunotherapeutic agent antagonizing CD47 interaction with SIRPα to increase depletion of NK cells.

36. The method of any preceding claim, wherein the patient is a human.

37. Use of an immunotherapeutic agent antagonizing CD47 interaction with SIRPα in the manufacture of a medicament for depleting endogenous T-cells or NK-cells before administration of genetically engineered T-cells or NK-cells in combination with an immunotherapeutic agent binding to a T-cell or NK cell antigen.

38. Use of immunotherapeutic agent binding to a T-cell or NK cell antigen in the manufacture of a medicament for depleting endogenous T-cells or NK-cells before administration of genetically engineered T-cells or NK-cells in combination with an immunotherapeutic agent antagonizing CD47 interaction with SIRPα.

39. The use of claim 37 or 38 in accordance with the method of any of claims 2-36.


SEQUENCE LISTING (48 sequences)

SEQ ID NO: 1 - protein, 5 aa, Artificial Sequence
  5F9 heavy chain CDR1
NYNMH

SEQ ID NO: 2 - protein, 17 aa, Artificial Sequence
  5F9 heavy chain CDR2
TIYPGNDDTSYNQKFKD

SEQ ID NO: 3 - protein, 8 aa, Artificial Sequence
  5F9 heavy chain CDR3
GGYRAMDY

SEQ ID NO: 4 - protein, 16 aa, Artificial Sequence
  5F9 light chain CDR1
RSSQSIVYSNGNTYLG

SEQ ID NO: 5 - protein, 7 aa, Artificial Sequence
  5F9 light chain CDR2
KVSNRFS

SEQ ID NO: 6 - protein, 9 aa, Artificial Sequence
  5F9 light chain CDR3
FQGSHVPYT

SEQ ID NO: 7 - protein, 117 aa, Artificial Sequence
  Humanized antibody hu5F9-vh1
QVQLVQSGAEVKKPGASVKVSCKASGYTFTNYNMHWVRQAPGQGLEWIGTIYPGNDDTSYNQKFKDKATLTADKSTSTAYMELSSLRSEDTAVYYCARGGYRAMDYWGQGTLVTVSS

SEQ ID NO: 8 - protein, 117 aa, Artificial Sequence
  Humanized antibody hu5F9-vh2
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 9 - protein, 117 aa, Artificial Sequence
  Humanized antibody hu5F9-vh3
QVQLVQSGAEVKKPGASVKVSCKASGYTFTNYNMHWVRQAPGQRLEWIGTIYPGNDDTSYNQKFKDRATLTADKSASTAYMELSSLRSEDTAVYYCARGGYRAMDYWGQGTLVTVSS

SEQ ID NO: 10 - protein, 112 aa, Artificial Sequence
  Humanized antibody hu5F9-vl1
DVVMTQSPLSLPVTPGEPASISCRSSQSIVYSNGNTYLGWYLQKPGQSPKLLIYKVSNRFSGVPDRFSGSGSGTDFTLKISRVEAEDVGVYHCFQGSHVPYTFGGGTKVEIK

SEQ ID NO: 11 - protein, 112 aa, Artificial Sequence
  Humanized antibody hu5F9-vl2
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 12 - protein, 112 aa, Artificial Sequence
  Humanized antibody hu5F9-vl3
DVVMTQSPLSLPVTPGEPASISCRSSQSIVYSNGNTYLGWYLQKPGQSPQLLIYKVSNRFSGVPDRFSGSGSGTDFTLKISRVEAEDVGVYHCFQGSHVPYTFGQGTKLEIK

SEQ ID NO: 13 - protein, 5 aa, Artificial sequence
  1H9 CDR-H1
SYWIT

SEQ ID NO: 14 - protein, 17 aa, Artificial sequence
  1H9 CDR-H2
DIYPGSGSTNHIEKFKS

SEQ ID NO: 15 - protein, 11 aa, Artificial sequence
  1H9 CDR-H3
GYGSSYGYFDY

SEQ ID NO: 16 - protein, 11 aa, Artificial sequence
  1H9 CDR-L1
RASENIYSYLA

SEQ ID NO: 17 - protein, 7 aa, Artificial sequence
  1H9 CDR-L2
TAKTLAE

SEQ ID NO: 18 - protein, 9 aa, Artificial sequence
  1H9 CDR-L3
QHQYGPPFT

SEQ ID NO: 19 - protein, 120 aa, Artificial sequence
  Humanized 1H9 VH
QVQLVQSGAEVKKPGASVKVSCKASGYTFTSYWITWVKQAPGQGLEWIGDIYPGSGSTNHIEKFKSKATLTVDTSISTAYMELSRLRSDDTAVYYCATGYGSSYGYFDYWGQGTLVTVSS

SEQ ID NO: 20 - protein, 107 aa, Artificial sequence
  Humanized 1H9 VL
DIQMTQSPSSLSASVGDRVTITCRASENIYSYLAWYQQKPGKAPKLLIYTAKTLAEGVPSRFSGSGSGTDFTLTISSLQPEDFATYYCQHQYGPPFTFGQGTKLEIK

SEQ ID NO: 21 - protein, 5 aa, Artificial sequence
  3C2 CDR-H1
SYWMH

SEQ ID NO: 22 - protein, 17 aa, Artificial sequence
  3C2 CDR-H2
NIDPSDSDTHYNQKFKD

SEQ ID NO: 23 - protein, 10 aa, Artificial sequence
  3C2 CDR-H3
GYSKYYAMDY

SEQ ID NO: 24 - protein, 16 aa, Artificial sequence
  3C2 CDR-L1
RSSQSIVHSYGNTYLE

SEQ ID NO: 25 - protein, 7 aa, Artificial sequence
  3C2 CDR-L2
KVSNRFS

SEQ ID NO: 26 - protein, 9 aa, Artificial sequence
  3C2 CDR-L3
FQGSHVPYT

SEQ ID NO: 27 - protein, 119 aa, Artificial sequence
  Humanized 3C2 VH
QVQLVQSGAEVKKPGASVKVSCKASGYTFTSYWMHWVRQAPGQGLEWMGNIDPSDSDTHYNQKFKDRVTMTRDTSTSTVYMELSSLRSEDTAVYYCARGYSKYYAMDYWGQGTLVTVSS

SEQ ID NO: 28 - protein, 112 aa, Artificial sequence
  Humanized 3C2 VL
DIVMTQTPLSLSVTPGQPASISCRSSQSIVHSYGNTYLEWYLQKPGQSPQLLIYKVSNRFSGVPDRFSGSGSGTDFTLKISRVEAEDVGVYYCFQGSHVPYTFGQGTKLEIK

SEQ ID NO: 29 - protein, 449 aa, Artificial sequence
  Humanized 1H9 HC (full-length)
QVQLVQSGAEVKKPGASVKVSCKASGYTFTSYWITWVKQAPGQGLEWIGDIYPGSGSTNHIEKFKSKATLTVDTSISTAYMELSRLRSDDTAVYYCATGYGSSYGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 214 aa, Artificial sequence
  Humanized 1H9 LC (full-length)
DIQMTQSPSSLSASVGDRVTITCRASENIYSYLAWYQQKPGKAPKLLIYTAKTLAEGVPSRFSGSGSGTDFTLTISSLQPEDFATYYCQHQYGPPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 448 aa, Artificial sequence
  Humanized 3C2 HC (full-length)
QVQLVQSGAEVKKPGASVKVSCKASGYTFTSYWMHWVRQAPGQGLEWMGNIDPSDSDTHYNQKFKDRVTMTRDTSTSTVYMELSSLRSEDTAVYYCARGYSKYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 219 aa, Artificial sequence
  Humanized 3C2 LC (full-length)
DIVMTQTPLSLSVTPGQPASISCRSSQSIVHSYGNTYLEWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 5 aa, Artificial sequence
  9B11 CDR-H1
DYYIH

SEQ ID NO: 34 - protein, 17 aa, Artificial sequence
  9B11 CDR-H2
RIDPEDGETKYAPKFQG

SEQ ID NO: 35 - protein, 5 aa, Artificial sequence
  9B11 CDR-H3
GGFAY

SEQ ID NO: 36 - protein, 11 aa, Artificial sequence
  9B11 CDR-L1
ASSSVSSSYLY

SEQ ID NO: 37 - protein, 7 aa, Artificial sequence
  9B11 CDR-L2
STSNLAS

SEQ ID NO: 38 - protein, 9 aa, Artificial sequence
  9B11 CDR-L3
HQWSSHPYT

SEQ ID NO: 39 - protein, 114 aa, Artificial sequence
  9B11 VH
EVQLQQSGAELVKPGASVKLSCTASGFNIKDYYIHWVKQRTEQGLEWIGRIDPEDGETKYAPKFQGKATITADTSSNTAYLQLNSLTSEDTAVYSCAKGGFAYWGQGTLVTVSA

SEQ ID NO: 40 - protein, 108 aa, Artificial sequence
  9B11 VL
QIVLTQSPAIMSASPGEKVTLTCSASSSVSSSYLYWYQQKPGSSPKLWIYSTSNLASGVPARFSGSGSGTSYSLTISSMEAEDAASYFCHQWSSHPYTFGGGTKLEIK

SEQ ID NO: 41 - protein, 5 aa, Artificial sequence
  7E11 CDR-H1
SYWMH

SEQ ID NO: 42 - protein, 17 aa, Artificial sequence
  7E11 CDR-H2
NIDPSDSDTHYNQKFKD

SEQ ID NO: 43 - protein, 12 aa, Artificial sequence
  7E11 CDR-H3
SYGNYGENAMDY

SEQ ID NO: 44 - protein, 16 aa, Artificial sequence
  7E11 CDR-L1
RSSQSIVHSYGNTYLE

SEQ ID NO: 45 - protein, 7 aa, Artificial sequence
  7E11 CDR-L2
KVSNRFS

SEQ ID NO: 46 - protein, 9 aa, Artificial sequence
  7E11 CDR-L3
FQGSHVPFT

SEQ ID NO: 47 - protein, 120 aa, Artificial sequence
  7E11 VH
QVKLQESGAELVRPGSSVKLSCKASGYTFTSYWMHWVKQRPIQGLEWIGNIDPSDSDTHYNQKFKDKATLTVDNSSSTAYMQLSSLTSEDSAVYYCASYGNYGENAMDYWGQGTSVTVSS

SEQ ID NO: 48 - protein, 112 aa, Artificial sequence
  7E11 VL
DILMTQTPLSLPVSLGDQASISCRSSQSIVHSYGNTYLEWYLQKPGQSPKLLIYKVSNRFSGVPDRFSGSGSGTDFTLKISRVEAEDLGVYYCFQGSHVPFTFGSGTKLEIK